(19) United States
(12) Patent Application Publication
(10) Pub. No.: US 2023/0372236 A1
(43) Pub. Date: Nov. 23, 2023

(54) DRUG CONTAINING DISSOLVABLE OCULAR INSERTS AND METHOD OF USING SAME

(71) Applicant: Novartis AG, Basel (CH)

(72) Inventors: Stephen John COLLINS, Fort Worth, TX (US); Howard Allen KETELSON, Dallas, TX (US); Walter R. LAREDO, Fort Worth, TX (US)

(21) Appl. No.: 18/248,000

(22) PCT Filed: Oct. 5, 2021
(86) PCT No.: PCT/IB2021/059125

§ 371 Date: Apr. 5, 2023

Related U.S. Application Data
(60) Provisional application No. 63/088,614, filed on Oct. 7, 2020.
(60) Provisional application No. 63/151,149, filed on Feb. 19, 2021.

(51) Int. Cl. A61K 9/00; A61K 31/517; A61K 47/36; A61K 47/32; A61K 47/10; A61K 9/70; A61P 27/02

(52) U.S. Cl. CPC A61K 9/0051; A61K 31/517; A61K 47/36; A61K 47/32; A61K 47/10; A61K 9/7007; A61P 27/02

(57) ABSTRACT

Polymeric eye inserts are provided that may be dissolvable when placed in the cul-de-sac of the eye. These inserts may contain one or more polymers as well as a softener/plasticizer so that, when inserted into the eye, they may absorb tears, and dissolve and slowly release lubricant into the tear film to lubricate and protect the ocular surface for an extended duration of time. Increased retention time on the ocular surface for longer lasting relief may reduce dosing frequency and patient burden typically associated with topical drop usage. These polymeric eye inserts also may include one or more pharmaceutically active agents.

The inventions described herein were made pursuant to a Joint Research Agreement between Novartis Pharma AG and Alcon Inc.

### FIELD OF THE DISCLOSURE

The present disclosure generally relates to polymeric eye insert technology, and more particularly to dissolvable polymeric eye inserts that release lubricants and drugs into the eye (including, but not limited to the anterior and posterior segments) for an extended duration of time compared to topical drop dosage forms.

### BACKGROUND

Many ophthalmic formulations comprise compounds that provide lubricity and other desirable properties. When these formulations are instilled in the eye, the properties of such compounds can prevent undesirable problems such as bioadhesion and the formation of friction-induced tissue damage, as well as encourage the natural healing and restoration of previously damaged tissues.

Compliance with administration of topically applied ophthalmic formulations such as liquids, ointments, gels, sprays is often poor, specifically for the treatment of dry eye, allergy, infection and slowly progressing diseases, such as glaucoma, requiring multiple applications per day to lubricate and deliver a drug to the eye. Exposure to topically administered aqueous formulations is often driven by the short retention time of the formulation on the ocular surface, which can be less than 25 minutes following instillation. Paugh et al., Optom Vis Sci. 2008 August; 85(8):725-31. Typical aqueous formulations for ocular use may be diluted or washed from the ocular surface within minutes, introduce variability in the usage, or result in less accurate and precise dosages administered to the eye. Accordingly, there is a need to reduce treatment burden and improve compliance.

Ointments and gels, which are highly viscous and usually reside in the eye longer than a liquid can provide for more accurate administration. However, they can also interfere with a patient's vision and may require, at a minimum, dosing 2-3 times per day. For these and other reasons the rate of discontinuation of use can be very high. Swanson, M., J. Am. Optom. Assoc., 2011; 10:649-6.

Inserts, both bioerodible and non-bioerodible, are also available and allow for less frequent administration. Pescina S et al., Drug Dev Ind Pharm; 2017 May 7:1-8; Karthikeyan, M B et al., Asian J. Pharmacol; 2008; October-December 192-200. These inserts, however, require complex and detailed preparation and can be uncomfortable to the patient. An additional problem with non-bioerodible inserts is that they must be removed after use. However, with proper use and adequate patient education, inserts can be an effective and safe treatment choice for dry eye patients.

Hydroxypropyl cellulose ophthalmic inserts such as LACRISERT® (Aton Pharmaceuticals Inc.) have been used for dry eye patients. These inserts are translucent cellulose-based rods measuring 1.27 mm in diameter and 3.5 mm in length. Each of these inserts contains 5 mg of hydroxypropyl cellulose, with no preservatives or other ingredients. The medication is administered by placing a single insert into the inferior cul-de-sac of the eye beneath the base of the tarsus. These inserts are indicated particularly for patients who continue to have dry eye symptoms following an adequate trial therapy with artificial tears. They also are indicated for patients with keratoconjunctivitis sicca, exposure keratitis, decreased corneal sensitivity, and recurrent corneal erosions. Several studies have been performed to evaluate the efficacy of HPC ophthalmic inserts. (Luchs, J, et al., Cornea, 2010; 29:1417-1427; Koffler B, et al., Eye Contact Lens; 2010; 36:170-176; McDonald M, et al., Trans Am Ophthalmol. Soc., 2009; 107:214-221; Wander A, and Koffler B, Ocul Surf 2009 July; 7(3):154-62).

However, there also are challenges in using these types of inserts. For example, LACRISERT® inserts tend to dissolve slowly and can remain in the eye even after 15-20 hours. The rod is hard and inelastic with edges due to rod-shaped design. The slow dissolving properties coupled with the rod hardness and design may lead to side effects including blurred vision, foreign body sensation and/or discomfort, ocular irritation or hyperemia, hypersensitivity, photophobia, eyelid edema, and caking or drying of viscous material on eyelashes. The most common side effect of these hydroxypropyl cellulose ophthalmic inserts is blurred vision due to the long retention time of the insert. Thus, additional approaches are needed to develop ocular inserts that are comfortable and improve patient compliance and provide prolonged drug delivery to the eye.

### SUMMARY

In one embodiment, the invention described herein is a polymeric eye insert, comprising:

one or more mucoadhesive polymers that are biocompatible with the ocular surface and tear film of the eye; and
one or more pharmaceutically active agent.

In one embodiment, upon insertion of the polymeric eye insert in the cul-de-sac of the eye, the thickness of the tear film increases for at least 30 minutes post-insertion. In some embodiments, the one or more mucoadhesive polymers in the polymeric eye insert are selected from the group consisting of hyaluronic acid or salts thereof, hydroxypropylmethylcellulose (HPMC), methylcellulose, tamarind seed polysaccharide (TSP), guar, hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and combinations thereof. In particular embodiments, the one or more mucoadhesive polymers are HP guar, hyaluronic acid, or sodium hyaluronate.

In some embodiments, the one or more mucoadhesive polymers are present in an amount of from about 50% to about 99% w/w, about 60% to about 95% w/w, about 70% to about 90% w/w, or about 80% to about 90% w/w of the polymeric eye insert.

In some embodiments of the present invention, the polymeric eye insert further includes a plasticizer or softener. In some embodiments, the plasticizer or softener is selected from the group consisting of polyethylene glycol (PEG), a PEG derivative, water, Vitamin E, and triethyl citrate. In particular embodiments, the plasticizer or softener is PEG.

In some embodiments, the plasticizer or softener is present in an amount of from about 2% to about 30% w/w, about 5% to about 25% w/w, about 5% to about 20% w/w, or about 5% to about 15% w/w of the polymeric eye insert.

In some embodiments of the present invention, the polymeric eye insert is comprised of approximately 40% HP guar, approximately 10% PVP, approximately 40% sodium hyaluronate, and approximately 10% PEG.

In some embodiments, polymeric eye insert further includes 1-200 ppm menthol. In particular embodiments, the polymer eye insert further includes 20-100 ppm menthol.

In some embodiments of the present invention, the one or more pharmaceutically active agents in the polymeric eye insert is selected from the group consisting of drugs used to treat the eye. In particular embodiments, the one or more pharmaceutically active agents is present in the polymeric eye insert at a concentration of about 0.01-10% w/w or in an amount of from about 0.01 mg to about 10 mg. In some embodiments, the pharmaceutically active agent is 4-(7-hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 4-(7-hydroxy-2-isopropyl-4-oxo-4H-quinazolin-3-yl)-benzonitrile or salts thereof. In particular embodiments, the 4-(7-hydroxy-2-isopropyl-4-oxo-4H-quinazolin-3-yl)-benzonitrile or salts thereof is present in an amount of about 0.5% w/w, about 1.0% w/w, about 1.5% w/w, about 2.0% w/w, about 2.5% w/w, about 3.0% w/w, or about 3.5% w/w or wherein the 4-(7-hydroxy-2-isopropyl-4-oxo-4H-quinazolin-3-yl)-benzonitrile or salts thereof is present in an amount from about 0.01 mg to about 5 mg, from about 0.01 mg to about 1 mg, from about 0.01 mg to about 0.5 mg, from about 0.01 mg to about 0.1 mg, from about 0.01 mg to about 0.08 mg per polymeric eye insert. In particular embodiments, the polymeric eye insert includes 4-(7-hydroxy-2-isopropyl-4-oxo-4H-quinazolin-3-yl)-benzonitrile or salts thereof in an amount of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.15 mg, about 0.2 mg, about 0.25 mg, about 0.3 mg, about 0.35 mg, about 0.4 mg, about 0.45 mg, or about 0.5 mg per polymeric eye insert.

In some embodiments of the present invention, the 4-(7-hydroxy-2-isopropyl-4-oxo-4H-quinazolin-3-yl)-benzonitrile in the polymeric eye insert is present as crystal form B, characterized by an X-ray diffraction pattern having three or more peaks at 2θ values selected from 9.3° 10.6° 14.4°±0.2 °2 θ, when recorded using CuK_{α} radiation.

In some embodiments, upon insertion of the polymeric eye insert into the eye, the tear film thickness of the eye does not return to pre-insertion thickness until approximately two hours after insertion. In some embodiments, upon insertion in the cul-de-sac of the eye, tear film thickness of the eye increases up to at least two hours post-insertion.

In some embodiments, the polymeric eye insert described herein has a shape as a film, a rod, a sphere, or an irregular shape having a maximum size in any single dimension of 5-7 mm. In particular embodiments, the polymeric eye insert described herein has a shape that is suitable for insertion into the eye.

In some embodiments, the polymeric eye insert has a circular shape about 5 mm in diameter, a thickness of 50-400 μm, and a water content of 1% to 50% w/w.

In some embodiments, the polymeric eye insert has a water content of about 1% to about 50% w/w, in particular about 30-40% w/w.

In some embodiments, the polymeric eye insert has a circular shape about 5 mm in diameter, a thickness of 50-400 μm, and a water content of 1% to 50% w/w.

In some embodiments, the polymeric eye insert has a thickness of about 150-250 μm, and a water content of 30 to 50% w/w.

In some embodiments, the polymeric eye insert has a thickness of about 150-250 μm, and a water content of 30 to 50% w/w.

In some embodiments of the polymeric eye insert described herein that include HP guar and sodium hyaluronate, the HP guar has a molecular weight of 2 to 3 million Daltons and sodium hyaluronate has a molecular weight of 0.1 to 1 million Daltons.

In some embodiments of the polymeric eye insert described herein, upon insertion of the polymeric eye insert in the cul-de-sac of the eye, the thickness of the tear film increases for at least 30 minutes post-insertion.

In some embodiments of the polymeric eye insert described herein, the polymeric eye insert dissolves over an extended duration of time after insertion into the eye. In some embodiments, the polymeric eye insert dissolves within about 1 to 2 hours, 1 to 3 hours, 1 to 4 hours, 1 to 5 hours, 1 to 6 hours, 1 to 7 hours, 1 to 8 hours, 1 to 9 hours, or 1 to 10 hours after insertion into the eye. In some embodiments, the polymeric eye insert dissolves within about 60 to 90 minutes after insertion into the eye. In some embodiments, the polymeric eye insert dissolves in about 60, about 90, about 120 minutes, about 3 hours, about 4 hours, about 5 hours, about 6 hours, about 7 hours, about 8 hours, about 9 hours, or about 10 hours after insertion into the eye.

In some embodiments, the polymeric eye insert has a thickness of about 50-250 μm, particularly of about 70-150 μm, when dissolved within about 60 to 120 minutes after insertion into the eye. In particular embodiments, the polymeric eye insert has a thickness of about 90 μm when dissolved within about 60 to 120 minutes after insertion into the eye.

In some embodiments of the polymeric eye insert described herein, the polymeric eye insert has a Young's modulus of less than about 0.6 MPa as determined using the Instron test method. In particular embodiments, the polymeric eye insert has a Young's modulus of about 0.2-0.5 MPa as determined using the Instron test method. In further embodiments, the polymeric eye insert has a Young's modulus of less than about 0.6 MPa, particularly about 0.2-0.5 MPa, such that the % elongation at break is between about 120-150% as determined using the Instron test method.

In some embodiments, the present invention provides a method of providing extended drug delivery or prolonging exposure of a pharmaceutically active agent to the eye, by administering the polymeric eye insert as described herein, to a patient in need thereof. In particular embodiments, the pharmaceutically active agent is as described herein.

In some embodiments, the present invention provides a method of making a polymeric eye insert as described herein, comprising the step of preparing a mixture comprising the one or more mucoadhesive polymers, optionally, the plasticizer or softener, and one or more pharmaceutically active agent in a suitable first solvent. In particular embodiments, the mixture is stirred and/or sonicated. In yet particular embodiments, a pharmaceutically active agent is added during preparation of the polymeric eye insert. The pharmaceutically active agent may be added as a solution in an acceptable solvent or as in undissolved form.

In some embodiments, the invention described herein is a method of treating or reducing the symptoms of an ocular disorder, which comprises applying the polymeric eye insert described herein to the cul-de-sac of the eye. In some embodiments, the ocular disorder is selected from the group consisting of ocular pain, myopia, presbyopia, dry eye, glaucoma, allergy, inflammation, dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, conjunctivochalasis, keratopathis (including herpetic keratopathy, filamentary keratopathy, band or bullous keratopathy, exposure keratopathy), keratitis (including herpes simplex virus keratitis), iritis, and episclentis. In particular embodiments, the ocular surface pain is due to dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, patients recovering from neurotrophic keratitis, or ocular pain persisting for at least three months after photorefractive keratectomy (PRK) surgery or laser-assisted in situ keratomileusis (LASIK) surgery.

In some embodiments, the present invention relates to a polymeric eye insert, the insert comprising:

one or more mucoadhesive polymers that are biocompatible with the ocular surface and tear film of the eye; and

one or more pharmaceutically active agent, wherein a pharmacokinetic profile of the active agent in rabbit is achieved with at least a 5 (6, 7, 8, 9, 10) fold lower dose of the active agent in the eye insert relative to the pharmacokinetic profile in rabbit studies of the active agent not formulated in an eye insert (e.g., in solution or suspension).

It will be appreciated that any of the preceding embodiments in this section and hereafter may be combined to produce further embodiments of the present invention.

### BRIEF DESCRIPTION OF THE DRAWINGS

For a more complete understanding of this disclosure, reference is now made to the following description, taken in conjunction with the accompanying drawings, in which:

FIG. 1 depicts placement of a polymeric eye insert according to embodiments of the present disclosure;

FIGS. 2A-2C depict tear film measurements for SYSTANE® ULTRA eye drops pre-dose (FIG. 2A), immediately post-dose (FIG. 2B) and 5 minutes post-dose (FIG. 2C);

FIGS. 3A-3C depict tear film measurements for GENTEAL® gel drops pre-dose (FIG. 3A), immediately post-dose (FIG. 3B) and 5 minutes post-dose (FIG. 3C);

FIGS. 4A-4E depict tear film measurements for PROVISC® injectable pre-dose (FIG. 4A), immediately post-dose (FIG. 4B), 5 minutes post-dose (FIG. 4C), 10 minutes post-dose (FIG. 4D) and 20 minutes post-dose (FIG. 4E);

FIGS. 5A-5I reflect tear film measurements associated with insertion of a polymeric eye insert according to embodiments of the present disclosure;

FIG. 6A reflects mean tear film measurements using polymeric eye inserts according to an embodiment of the present disclosure;

FIG. 6B reflects tear film measurements by individual animal according to an embodiment of the present disclosure;

FIG. 6C reflects tear film measurements based on location in the eye including the bottom of the eye, top of the eye, temporal and nasal measurements according to an embodiment of the present disclosure;

FIG. 7A reflects the dynamic change of tear film thickness with respect to polymeric eye inserts according to embodiments of the present disclosure;

FIG. 7B reflects tear film measurements by location (apex, nasal, temporal, top, and bottom) for polymeric eye inserts according to embodiments of the present disclosure;

FIG. 8 reflects mean GENTEAL® gel tear film measurements for the right and left eye;

FIG. 9 reflects tear film thickness data as a function of elapsed time post-dose; and

FIGS. 10A-10C illustrate various polymeric eye insert shapes and characteristics according to embodiments of the present disclosure.

FIG. 11 illustrates the results from the primary outcome measure (comfort rating) of two embodiments (thick insert and thin insert) according to embodiments of the present disclosure;

FIG. 12 illustrates the results from a secondary outcome measure (visual blur) of two embodiments (thick insert and thin insert) according to embodiments of the present disclosure;

FIG. 13 illustrates the results from the assessment of ocular insert dissolution of embodiments of the present disclosure;

FIG. 14 illustrates the results from a secondary outcome measure (NITBUT) of two embodiments (thick insert and thin insert) according to the present disclosure;

FIG. 15 illustrates the results from a secondary outcome measure (tear meniscus height) of two embodiments (thick insert and thin insert) according to the present disclosure;

FIG. 16 illustrates results from the ocular irritation question for two embodiments (thick insert and thin insert) according to the present disclosure;

FIG. 17 illustrates results from the ocular dryness question for two embodiments (thick insert and thin insert) according to the present disclosure;

FIG. 18 illustrates results from the ocular burning/stinging question for two embodiments (thick insert and thin insert) according to the present disclosure;

FIG. 19 illustrates results from the ocular itching question for two embodiments (thick insert and thin insert) according to the present disclosure;

FIG. 20 illustrates the XRD spectra of dry films containing 4-(7-Hydroxy-2-isopropyl-4-oxo-4H-quinazolin-3-yl)-benzonitrile that were prepared according to the sonication/stirring method and ethanolic solution method according to the present disclosure. Spectrum A shows the XRD of 4-(7-Hydroxy-2-isopropyl-4-oxo-4H-quinazolin-3-yl)-benzonitrile crystal form B. Spectrum B represents the ocular film with 2% 4-(7-Hydroxy-2-isopropyl-4-oxo-4H-quinazolin-3-yl)-benzonitrile, prepared according to the ethanol solution method. Spectrum C represents the ocular film without the presence of 4-(7-Hydroxy-2-isopropyl-4-oxo-4H-quinazolin-3-yl)-benzonitrile. Spectrum D represents the ocular film with 2% 4-(7-Hydroxy-2-isopropyl-4-oxo-4H-quinazolin-3-yl)-benzonitrile as crystal form B, prepared according to the sonication/stirring method.

### DETAILED DESCRIPTION

The language “effective amount” of the compounds described herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal, and the ability of the therapeutic compounds of the present disclosure to treat the ocular surface disorder and/or symptoms thereof in the mammal.

The phrase “ophthalmically compatible” refers to formulations, polymers and other materials and/or dosage forms which are suitable for use in contact with the ocular tissues of human beings and animals without excessive toxicity, irritation, allergic response, or other problem or complication, commensurate with a reasonable benefit/risk ratio.

As used herein, the term “treat”, “treating” or “treatment” in connection to a disease or disorder refers in some embodiments, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both.

As used herein, the term “subject” or “patient” refers to human and non-human mammals, including but, not limited to, primates (e.g., non-human primates), rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment. In particular embodiments, the subject is an adult human at least about 18 years of age. In particular embodiments, the subject is an adult human from about 18 to about 75 years of age. In some embodiments, the subject is a human child up to about 18 years of age.

As used herein, “ocular surface” refers to the outer surface of the eye, which anatomically comprises the cornea (with epithelium, bowman layer, stroma, descement membrane, endothelium), conjunctiva, cul de sac, and the corneo-scleral junction, i.e., limbus.

As used herein, ocular administration includes administration to all parts of the eye including all parts of the ocular surface such as the cornea, conjunctiva, the cul de sac and the corneo-scleral junction, i.e., limbus.

As used herein, “pain” refers to constant or intermittent sensation of actual pain described as but not limited to stabbing, dull, sharp, or ache. Pain may also refer to similar related descriptors such as but not limited to burning, stinging, grittiness, foreign body sensation, dryness, sandy, tired, itchy, irritated, sensitivity to light.

As used herein, “ocular surface pain” refers to pain on the surface of the eye, e.g., cornea. Ocular pain may be nociceptic pain, which is generally caused by external physical or chemical damaging stimuli such as corneal surgery, inflammation, or other damage to the corneal surface. Ocular pain may also result from neuropathic pain, which may occur due to direct damage to the neurons of the body, resulting in messages of pain being sent to the central nervous system and brain regardless of the presence of noxious stimuli. As used herein “ocular surface pain” includes both nociceptic pain and neuropathic pain.

As used herein, the term “visual analog scale” (VAS) is a measure of pain intensity where a subject typically marks a place on a scale that aligns with their level of pain. The pain is marked in a range of “no pain” (score of 0) and “pain as bad as it could be” or “worst imaginable pain” (score of 100). See e.g., Hawker, et al., Arthritis Care & Research 63(11), pp. S240-S252 (November 2011). There are several other well-designed pain scales that may be used to help assess the extent of pain. The numerical rating scale (NRS) is often used, in which subjects use numbers to rate pain. The number scale may be from 1-10, or 1-100. The Wong-Baker FACES Pain Scale combines pictures and numbers for pain ratings. It can be used in children over the age of 3 and in adults. Six faces depict different expressions, ranging from happy to extremely upset. Each is assigned a numerical rating between 0 (smiling) and 10 (crying). The Verbal Pain Intensity Scale uses wordings on a scale to rate pain intensity: No Pain/Mild Pain/Moderate Pain/Severe Pain Very Severe Pain/Worst Possible Pain.

The Eye Sensation Scale is a specific pain scale was developed to measure ophthalmic pain severity. See Caudle L. E. et al., Optom Vis Sci. 2007 August; 84(8):752-62. In this scale, pain, discomfort or light sensitivity is typically measured by 5 category labels of “extreme,” “severe,” “moderate,” “mild,” or “none.”

The Ocular Pain Assessment Survey (OPAS) is a quantitative, multidimensional questionnaire, specifically designed for assessment of corneal and ocular surface pain and Quality of Life (QoL) changes. The OPAS assesses pain intensity, frequency of eye and non-eye pain, QoL changes, aggravating factors, associated factors, and symptomatic relief quantitative, allowing for monitoring of treatment responses. See Qazi et al., Ophthalmology July 123(7):1458-1468 (2016).

As used herein, the term “Visual Tasking Questionnaire” refers to a questionnaire that asks the subject to subjectively rate how much difficulty they have conducting certain activities that require a fixed or prolonged stare that may exacerbate ocular pain. The questionnaire also asks about coping mechanisms associated with the difficulties they experience during visual tasking activities.

As used herein, ocular hyperemia refers to redness of the ocular surface. Ocular hyperemia may be a clinical marker for inflammation and/or ocular irritation. Ocular hyperemia may be measured using the McMonnies scale, at values from 0 to 5, based on standard photographs.

As used herein, “placebo” refers to an ophthalmic formulation that includes all the components of the administered drug composition without the drug.

As used herein, the terms “about” or “approximately” refer to a range of values 10% of a specified value.

As used herein, a pharmaceutical composition is a composition suitable for pharmaceutical use. A composition suitable for pharmaceutical use may be sterile, homogeneous and/or isotonic.

As used herein, the term “hyaluronic acid or salts thereof” includes the corresponding metal salts of hyaluronic acid, including, for example, sodium hyaluronate (the sodium salt), potassium hyaluronate, zinc hyaluronate, magnesium hyaluronate, and calcium hyaluronate. It is well understood by one of ordinary skill in the art that the term “hyaluronic acid” includes the corresponding metal salts of the acid form.

Any chemical formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen, carbon, nitrogen, and oxygen, such as ³H, ¹¹C, ¹³C, ¹⁴C, and ¹⁵N. Accordingly, it should be understood that methods of the present invention can or may involve compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds can generally be prepared by conventional techniques known to those skilled in the art, e.g., using an appropriate isotopically-labeled reagents in place of the non-labeled reagent previously employed.

The present invention encompasses embodiments that include all pharmaceutically acceptable salts of the compounds useful according to the invention provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. For example, preferred pharmaceutically acceptable salts include, but are not limited to, mineral or organic acid salts of basic residues such as amines. For example, the salt can be a hydrochloride salt. Other examples of suitable salts can be found in U.S. Pat. No. 8,349,852, the content of which is hereby incorporated by its entirety.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, the present invention provides a formulation comprising one or more polymers. In some embodiments, the polymers are ophthalmically compatible. In some embodiments, the formulation is in the form of a polymeric eye insert that may be inserted in the lower eye lid (also known as the cul-de-sac) of the eye. In some embodiments, the formulation comprises hyaluronic acid (in acid or salt form), hydroxypropylmethylcellulose (HPMC), methylcellulose, tamarind seed polysaccharide (TSP), guar and derivatives thereof such as hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and/or combinations thereof. In particular embodiments, the formulation comprises hyaluronic acid, hydroxypropyl guar (HP guar), and a plasticizer, such as polyethylene glycol (PEG); however, other polymers and plasticizers/softeners. In some embodiments, the polymers are non-toxic and able to solubilize in eye fluids. In particular embodiments, the formulation comprises hyaluronic acid and HP guar.

In some embodiments of the formulations described herein comprise polymers in an amount of from about 50% to about 99% w/w, about 60% to about 95% w/w, about 70% to about 90% w/w, or about 80% to about 90% w/w by dry weight of the formulation. In particular embodiments, the mucoadhesive polymers are present in an amount of about 75%, about 80%, about 85%, about 90%, or about 95% w/w by dry weight of the formulation. The overall dry weight or mass of the formulation may be in the range of about 1 to about 10 mg, or about 2 to about 8 mg, and in particular embodiments may be from about 2.5 to about 5 mg.

In some embodiments of the present invention, the formulation further comprises a softener and/or plasticizer to facilitate fabrication of a softer, malleable delivery system and also provide improved comfort in insertion. A plasticizer may soften the material to provide for desirable dissolution rates. It should be appreciated softeners and/or plasticizers may be low or high-molecular weight compounds, including not limited to, polyethylene glycol (PEG) and derivatives thereof, water, Vitamin E, and triethyl citrate. In some embodiments, the plasticizer or softener is present in the formulation in an amount of from about 2% to about 30% w/w, about 5% to about 25% w/w, about 5% to about 20% w/w, or about 5% to about 15% w/w by dry weight of the formulation. In particular embodiments, the plasticizer or softener is present in an amount of about 5%, about 7%, about 10%, or 12%, or about 15%, w/w by dry weight of the formulation.

In some embodiments of the present invention, the formulation may have a water content of about 1% to about 50%. In particular embodiments, the formulation may have a water content of about 30-40%.

In particular embodiments, the formulation comprises sodium hyaluronate, HP guar, PVP and PEG, and one or more pharmaceutically active agent. In another embodiment, the formulation comprises approximately 40% HP guar, approximately 10% PVP, approximately 40% sodium hyaluronate and approximately 10% PEG, and one or more pharmaceutically active agent, wherein the formulation is in the form of a polymeric eye insert.

In some embodiments of the present invention, the formulation does not include a pharmaceutically active agent. In other embodiments, the formulation may include one or more pharmaceutically active agents. In some embodiments, the one or more pharmaceutically active agents is selected from the group consisting of known ophthalmically active agents. In particular embodiments, the pharmaceutically active agent is any drug used to treat the eye and surrounding tissues can be incorporated in the polymeric eye insert of this invention.

In particular embodiments of the present invention, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof, lipoic acid choline ester or salts thereof. In particular embodiments, the pharmaceutically active agent is 4-(7-Hydroxy-2-isopropyl-4-oxo-4H-quinazolin-3-yl)-benzonitrile.

Particular embodiments of the present invention provide a polymeric eye insert comprising an ocular lubricant containing one or more polymers. In an embodiment of the present invention, a polymeric eye insert may be comprised of hyaluronic acid, hydroxypropyl guar (HP guar), and a plasticizer, such as polyethylene glycol (PEG); however, other polymers and plasticizers/softeners may be used without departing from the present disclosure, as described herein. An insert according to embodiments of the present invention may be inserted in the eye, for example, the lower eye lid (also known as the cul-de-sac) of the eye, and upon insertion, the insert may rapidly absorb tears and dissolve to release the ocular lubricant into the tear film to lubricate and protect the ocular surface for an extended duration superior to previously known topical ophthalmic compositions. Pharmaceutically active agents also may be incorporated into polymeric eye inserts according to embodiments of the present disclosure. Insertion of a polymeric eye insert according to embodiments of the present disclosure may provide relief to the patient from symptoms of dry eye as well as other eye conditions.

In particular embodiments of the present invention, the biomaterial for forming a polymeric eye insert may be comprised of one or more polymers that are biocompatible with the ocular surface and tear film. Polymers that may be used in polymeric eye inserts according to embodiments of the present disclosure include, but are not limited to, hyaluronic acid (in acid or salt form), hydroxypropylmethylcellulose (HPMC), methylcellulose, tamarind seed polysaccharide (TSP), guar and derivatives thereof such as hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and/or combinations thereof. Suitably, the polymer is selected from the group consisting of hyaluronic acid or salts thereof, e.g., sodium hyaluronate, hydroxypropylmethylcellulose (HPMC), methylcellulose, guar, hydroxypropyl guar (HP guar), sodium carboxymethylcellulose, polyvinyl alcohol, polyvinyl pyrrolidine, and combinations thereof. Polymers used in inserts according to embodiments of the present disclosure should be non-toxic and able to solubilize in eye fluids to ensure that the insert is eventually cleared from the eye, generally over a 60-minute or 120 minute time frame. It should be appreciated that the polymer(s) selected should be mucoadhesive. It also should be appreciated that one or more polymers may be blended according to embodiments of the present disclosure. For example, in a particular embodiment of the present invention, hyaluronic acid (HA) may be blended with tamarind seed polysaccharide (TSP) because TSP has been shown to increase residence time of HA in aggregate blends and the blend has desired film mechanical and lubrication properties. In other embodiments of the present disclosure, as described in further detail below, hyaluronic acid may be combined with HP guar.

In some embodiments of the present invention, the one or more mucoadhesive polymers are present in an amount of from about 50% to about 99% w/w, about 60% to about 95% w/w, about 70% to about 90% w/w, or about 80% to about 90% w/w by dry weight of the polymeric eye insert. In particular embodiments, the mucoadhesive polymers are present in an amount of about 75%, about 80%, about 85%, about 90%, or about 95% w/w by dry weight of the polymeric eye insert. The overall dry weight or mass of the polymeric eye insert may be in the range of about 1 to about 10 mg, or about 2 to about 8 mg, and in particular embodiments may be from about 2.5 to about 5 mg.

In some embodiments of the present invention, a softener and/or plasticizer may be added to the one or more polymers to facilitate fabrication of a softer, malleable delivery system and also provide improved comfort in insertion. A plasticizer may soften the material to provide for desirable dissolution rates. It should be appreciated softeners and/or plasticizers may be low or high-molecular weight compounds, including not limited to, polyethylene glycol (PEG) and derivatives thereof, water, Vitamin E, and triethyl citrate.

In some embodiments of the present invention, the plasticizer or softener is present in an amount of from about 2% to about 30% w/w, about 5% to about 25% w/w, about 5% to about 20% w/w, or about 5% to about 15% w/w by dry weight of the polymeric eye insert. In particular embodiments, the plasticizer or softener is present in an amount of about 5%, about 7%, about 10%, or 12%, or about 15%, w/w by dry weight of the polymeric eye insert.

In some embodiments of the present invention, the polymeric eye insert may have a water content of about 1% to about 50%. In particular embodiments, the polymeric eye insert may have a water content of about 30-40%.

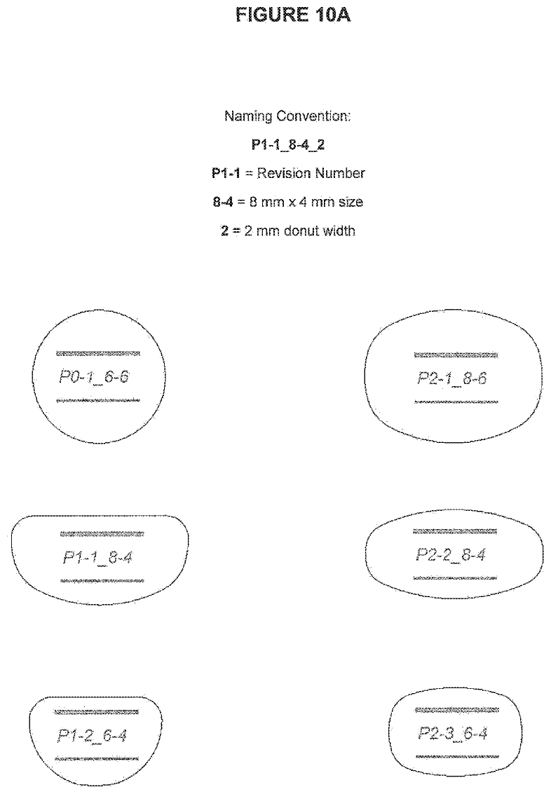

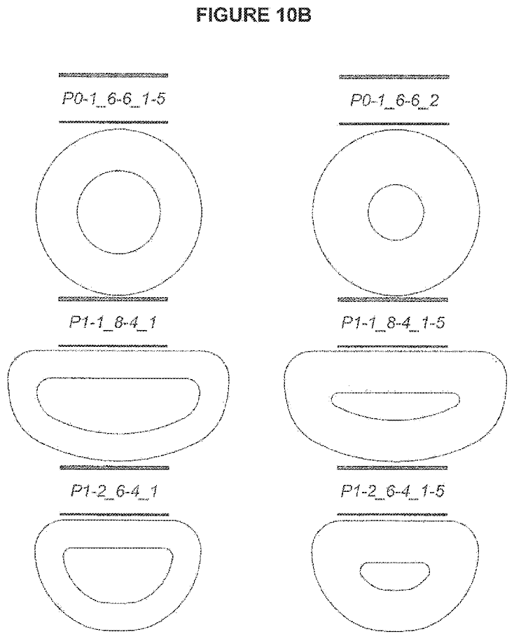

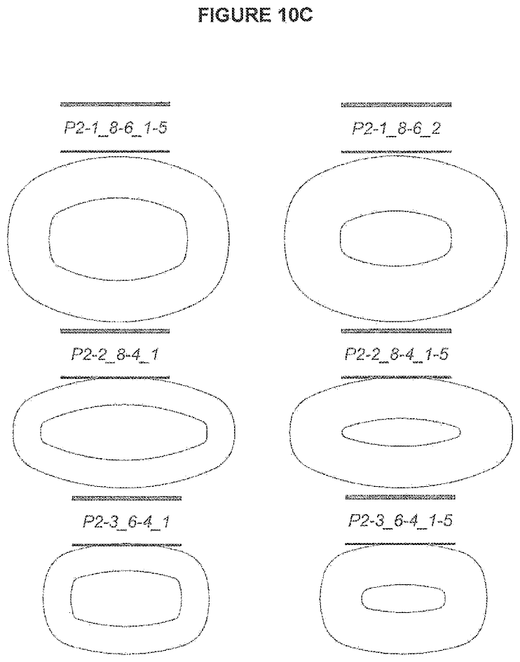

The polymeric eye insert may be of any size or shape suitable for administration to the eye. For example, the polymeric eye insert can have any shape such as square, rectangle, oval, circle, doughnut, semicircle, ¼ moon shape, and the like. Exemplary shapes include film, a rod, a sphere, or an irregular shape having a maximum size in any single dimension of 5-7 mm, or about 5-6 mm. Additional exemplary shapes are shown in FIGS. 10A-10C.

In some embodiments, the polymeric eye insert has a thickness of about 50-400 μm, about 100-300 μm, about 150-250 μm, about 200 μm, about 250 μm, or about 300 μm.

In particular embodiments of the present invention the polymeric eye insert has a thickness of about 150-250 μm, and a water content of 30 to 50% w/w.

In some embodiments of the present invention, the polymeric eye insert is inserted or placed on to the ocular surface. In particular embodiments, the polymeric eye insert is inserted into the cul de sac of the eye, or under the lower eyelid. As used herein, “insertion into the eye” includes administration to any part of the ocular surface, e.g., cornea or conjunctiva, including insertion into the cul de sac of the eye.

In some embodiments of the present invention, the polymeric eye insert does not include a pharmaceutically active agent. In other embodiments, the polymeric eye insert may include one or more pharmaceutically active agents. In some embodiments, the one or more pharmaceutically active agents is selected from the group consisting of ophthalmically active agent. In particular embodiments, the pharmaceutically active agent is any drug used to treat the eye and surrounding tissues can be incorporated in the polymeric eye insert of this invention.

In particular embodiments of the present invention, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, or salts thereof. In yet particular embodiments, the pharmaceutically active agent is 4-(7-Hydroxy-2-isopropyl-4-oxo-4H-quinazolin-3-yl)-benzonitrile or salts thereof. In some embodiments, the 4-(7-Hydroxy-2-isopropyl-4-oxo-4H-quinazolin-3-yl)-benzonitrile is present as crystal form B, characterized by an X-ray diffraction pattern having three or more peaks at 2θ values selected from 9.3° 10.6° 14.4°±0.2 °2 θ, typically, 9.3°, 10.6°, 14.4°, 15.5°, 17.9°, 19.9°, 23.4°±0.2 °2θ or more typically, 9.3, 10.6, 12.8, 14.4, 15.5, 17.9, 19.9, 21.3, 23.4, and 28.0±0.2 °2 θ, when recorded using CuK_{α} radiation. Polymorph form B is described in U.S. Pat. No. 8,349,852, incorporated by reference herein.

Thus, in an embodiment, the polymeric eye insert according to the present disclosure comprises one or more mucoadhesive polymers selected from the group consisting of hyaluronic acid (in acid or salt form, e.g., sodium hyaluronate), hydroxypropylmethylcellulose (HPMC), methylcellulose, tamarind seed polysaccharide (TSP), guar and derivatives thereof such as hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and/or combinations thereof; one or more pharmaceutically active agent, wherein the one or more mucoadhesive polymers are present in an amount of from about 50% to about 99% w/w, about 60% to about 95% w/w, about 70% to about 90% w/w, or about 80% to about 90% w/w by dry weight of the polymeric eye insert. In an embodiment, the mucoadhesive polymers are present in an amount of about 75%, about 80%, about 85%, about 90%, or about 95% w/w by dry weight of the polymeric eye insert. In an embodiment, a plasticizer or softener is additionally present. The plasticizer or softener may be selected from the group consisting of polyethylene glycol (PEG), a PEG derivative, water, Vitamin E, and triethyl citrate. Suitably, the plasticizer or softener is PEG. In a further embodiment, the plasticizer or softener is present in an amount of from about 2% to about 30% w/w, about 5% to about 25% w/w, about 5% to about 20% w/w, or about 5% to about 15% w/w by dry weight of the polymeric eye insert. In a further embodiment, the plasticizer or softener is present in an amount of about 5%, about 7%, about 10%, or 12%, or about 15%, w/w by dry weight of the polymeric eye insert.

In a further embodiment, the polymeric eye insert comprises sodium hyaluronate, HP guar, PVP and PEG, and one or more pharmaceutically active agent. In another embodiment, there is provided a polymeric eye insert, comprising approximately 40% HP guar, approximately 10% PVP, approximately 40% sodium hyaluronate and approximately 10% PEG, and one or more pharmaceutically active agent. Suitably, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone. More suitably, the pharmaceutically active agent is 4-(7-Hydroxy-2-isopropyl-4-oxo-4H-quinazolin-3-yl)-benzonitrile and is present as crystal form B, characterized by an X-ray diffraction pattern having three or more peaks at 2θ values selected from 9.3° 10.6° 14.4°+0.2 °2 θ, typically, 9.3°, 10.6°, 14.4°, 15.5°, 17.9°, 19.9°, 23.4°±0.2 °2θ or more typically, 9.3, 10.6, 12.8, 14.4, 15.5, 17.9, 19.9, 21.3, 23.4, and 28.0±0.2 °2 θ, when recorded using CuK_{α} radiation.

In some embodiments, the additional pharmaceutical active agent is present in the polymeric eye insert at a concentration of about 0.01-10% w/w. In alternative embodiments, the polymeric eye insert includes the additional pharmaceutical agent in amounts from about 0.01 mg to about 5 mg per polymeric eye insert. In some embodiments, the 4-(7-hydroxy-2-isopropyl-4-oxo-4H-quinazolin-3-yl)-benzonitrile or salts thereof is present in the polymeric eye insert at a concentration of about 0.5% w/w to about 3.5% w/w, based on dry weight of the polymeric eye insert. In particular embodiments, the 4-(7-hydroxy-2-isopropyl-4-oxo-4H-quinazolin-3-yl)-benzonitrile or salts thereof is present in the polymeric eye insert at a concentration of about 0.5% w/w, about 1.0% w/w, about 1.5% w/w, about 2.0% w/w, about 2.5% w/w, about 3.0% w/w, or about 3.5% w/w, based on the dry weight of the polymeric eye insert. In particular embodiments, the polymeric eye insert includes 4-(7-hydroxy-2-isopropyl-4-oxo-4H-quinazolin-3-yl)-benzonitrile or salts thereof in an amount of from about 0.01 mg to about 10 mg, from about 0.01 mg to about 5 mg, from about 0.01 mg to about 1 mg, from about 0.01 mg to about 0.5 mg, from about 0.01 mg to about 0.1 mg, from about 0.01 mg to about 0.08 mg per polymeric eye insert. In particular embodiments, the polymeric eye insert includes 4-(7-hydroxy-2-isopropyl-4-oxo-4H-quinazolin-3-yl)-benzonitrile or salts thereof in an amount of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.15 mg, about 0.2 mg, about 0.25 mg, about 0.3 mg, about 0.35 mg, about 0.4 mg, about 0.45 mg, or about 0.5 mg per polymeric eye insert. In some embodiments, the 4-(7-hydroxy-2-isopropyl-4-oxo-4H-quinazolin-3-yl)-benzonitrile or salts thereof is dissolved in the polymeric eye insert. In other embodiments, the 4-(7-hydroxy-2-isopropyl-4-oxo-4H-quinazolin-3-yl)-benzonitrile is present in the in the polymeric eye insert as a solid. In some embodiments, the 4-(7-hydroxy-2-isopropyl-4-oxo-4H-quinazolin-3-yl)-benzonitrile is present as an amorphous solid, or as crystal form B. In some embodiments, the 4-(7-hydroxy-2-isopropyl-4-oxo-4H-quinazolin-3-yl)-benzonitrile is present in the in the polymeric eye insert as crystal form A, characterized by an X-ray diffraction pattern having 3 or more, 4 or more, 5 or more, 6 or more, or 7 or more peaks at 2θ values selected from 7.2, 12.7, 13.3, 13.9, 14.5, 15.6, 18.1, 19.9, 21.4, 22.8, 25.1, 26.8, 27.8, 29.0±0.2 °2θ. In some embodiments, the 4-(7-hydroxy-2-isopropyl-4-oxo-4H-quinazolin-3-yl)-benzonitrile is present in the in the polymeric eye insert as crystal form C characterized by an X-ray diffraction pattern having 3 or more, 4 or more, 5 or more, 6 or more, or 7 or more peaks at 2θ values selected from 7.4, 14.1, 14.9, 16.4, 19.1, 24.8, 26.1, 28.4, 31.2±0.2 °2θ. In some embodiments, the 4-(7-hydroxy-2-isopropyl-4-oxo-4H-quinazolin-3-yl)-benzonitrile is present in the in the polymeric eye insert as crystal form E characterized by an X-ray diffraction pattern having 3 or more, 4 or more, 5 or more, 6 or more, or 7 or more peaks at 2θ values selected from 9.1, 11.9, 12.7, 13.8, 15.1, 16.7, 18.3, 21.3, 22.6, 24.4, 27.1, 27.7, 28.5, 37.8±0.2 °2θ. In some embodiments, the 4-(7-hydroxy-2-isopropyl-4-oxo-4H-quinazolin-3-yl)-benzonitrile is present in the in the polymeric eye insert as crystalline hydrate form F characterized by an X-ray diffraction pattern having 3 or more, 4 or more, 5 or more, 6 or more, or 7 or more peaks at 2θ values selected from 6.7, 11.9, 12.8, 14.4, 15.6, 16.3, 18.3, 19.5, 22.7, 23.9, 24.7, 25.6, 26.5, 29.2±0.2 °2θ. In some embodiments, the 4-(7-hydroxy-2-isopropyl-4-oxo-4H-quinazolin-3-yl)-benzonitrile is present in the in the polymeric eye insert as crystal form K characterized by an X-ray diffraction pattern having 3 or more, 4 or more, 5 or more, or 6 or more peaks at 2θ values selected from 5.3, 6.5, 10.5, 12.3, 17.2, 19.3, 22.4±0.2 °2θ. In some embodiments, the 4-(7-hydroxy-2-isopropyl-4-oxo-4H-quinazolin-3-yl)-benzonitrile is present in the in the polymeric eye insert as crystal form L, characterized by an X-ray diffraction pattern having 3 or more, 4 or more, 5 or more, 6 or more, or 7 or more peaks at 2θ values selected from 3.5, 7.1, 8.7, 10.6, 11.1, 12.2, 19.1, 21.1, 22.4, 23.4±0.2 °2θ. In some embodiments, the 4-(7-hydroxy-2-isopropyl-4-oxo-4H-quinazolin-3-yl)-benzonitrile is present in the in the polymeric eye insert as crystal form K′ characterized by an X-ray diffraction pattern having 3 or more, 4 or more, 5 or more, 6 or more, or 7 or more peaks at 2θ values selected from 5.3, 10.6, 12.3, 17.0, 17.3, 19.4, 20.3, 21.2, 22.5, 23.0, 24.8, 27.1, 32.0±0.2 °2θ. In some embodiments, the 4-(7-hydroxy-2-isopropyl-4-oxo-4H-quinazolin-3-yl)-benzonitrile is present in the in the polymeric eye insert as crystal form M characterized by an X-ray diffraction pattern having 3 or more, 4 or more, 5 or more, 6 or more, or 7 or more peaks at 2θ values selected from 11.1, 12.1, 18.5, 19.1, 20.1, 21.4, 21.7, 22.2, 23.1, 26.4, 273, 29.7 0.2 °2θ. In some embodiments, the 4-(7-hydroxy-2-isopropyl-4-oxo-4H-quinazolin-3-yl)-benzonitrile is present in the in the polymeric eye insert as Hydrate HB is characterized by an X-ray diffraction pattern having 3 or more, 4 or more, 5 or more, 6 or more, or 7 or more peaks at 2θ values selected from 6.6, 11.7,_12.2,_14.6, 15.8, 16.1, 18.5, 19.7, 20.9, 24.7, 26.5, 27.7, 29.3±0.2 °2θ. In some embodiments, the 4-(7-hydroxy-2-isopropyl-4-oxo-4H-quinazolin-3-yl)-benzonitrile is present in the in the polymeric eye insert as crystal form Q characterized by an X-ray diffraction pattern having 3 or more, 4 or more, 5 or more, 6 or more, or 7 or more peaks at 2θ values selected from 11.2, 12.2, 17.7, 18.5, 19.1, 20.1, 22.0, 22.5, 23.3, 24.2, 24.6, 26.5, 28.5±0.2 °2θ. In some embodiments, the polymeric eye insert may include partially dissolved 4-(7-hydroxy-2-isopropyl-4-oxo-4H-quinazolin-3-yl)-benzonitrile. All the peaks listed herein are as measured using CuK_{α} radiation. In some embodiments, the polymeric eye insert may include two or more polymorphic forms.

In some embodiments of the present invention, the polymeric eye insert dissolves over an extended duration of time after insertion into the eye. In some embodiments of the present invention, the polymeric eye insert dissolves within about two hours of insertion into the eye. In some embodiments, the polymeric eye insert dissolves in about 60 to 90 minutes after insertion into the eye. In particular embodiments, the polymeric eye insert dissolves in about 60, about 90, about 120 minutes, about 3 hours, about 4 hours, about 5 hours, about 6 hours, about 7 hours, about 8 hours, about 9 hours, or about 10 hours after insertion into the eye.

In some embodiments of the present invention, the polymeric eye insert maintains at least 80%, at least 90%, at least 92%, at least 94% at least 95%, at least 97%, or at least 98% of the original amount of pharmaceutically active agent after storage at about 25° C. for at least 1 month. In some embodiments of the present invention, the polymeric eye insert maintains at least 80%, at least 90%, at least 92%, at least 94% at least 95%, at least 97%, or at least 98% of the original amount of pharmaceutically active agent after storage at about 25° C. for at least 2 months or at least 3 months. In some embodiments of the present invention, the polymeric eye insert maintains at least 80%, at least 90%, at least 92%, at least 94% at least 95%, at least 97%, or at least 98% of the original amount of pharmaceutically active agent after storage at about 30° C. for at least 1 month. In some embodiments of the present invention, the polymeric eye insert maintains at least 80%, at least 90%, at least 92%, at least 94% at least 95%, at least 97%, or at least 98% of the original amount of pharmaceutically active agent after storage at about 30° C. for at least 2 months or at least 3 months. In one embodiment, the polymeric eye insert after storage at about 25° C. for at least 1 month or at least 2 months maintains strength as measured by Young's Modulus within 20%, within 15%, within 10%, within 5% of the original value. In particular embodiments, the polymeric eye insert after storage at about 30° C. for at least 1 month or at least 2 months maintains strength as measured by Young's Modulus within 20%, within 15%, within 10%, within 5% of the original value.

Polymeric eye inserts according to embodiments of the present invention may be made using various processing techniques, including but not limited to, compression molding and solution casting. Compression molding may be carried out at temperatures and pressures that do not change the material or lead to significant side reactions. For example, compression molding of partially hydrated polysaccharides may use a compressional force of approximately 5,000-12,000 pounds at approximately 200-300 degrees Celsius for approximately 1-2 minutes. Solution or film casting may be carried out using solvents and/or co-solvents that may provide homogeneous films with little to no defects. The solvent may be removed by air or vacuum drying, resulting in an insert material that may be free from residual solvents. For example, a 1% (w/v) aqueous solution of polymer (or blend) may be cast and then allowed to evaporate. The film may then be cut with an oval-shaped punch of desired size and geometry. While compression molding and solution/film casting have been described, it should be appreciated that other processing techniques may be used without departing from the present disclosure.

In one embodiment, the film casting method used was found to generate reproducible inserts and good structural integrity. In this embodiment, distilled water was placed in a 1 L Erlenmeyer flask followed by the addition of the polymer (s). The flask was placed in a sonicator and attached to an overhead mechanical stirrer. The mixture was sonicated and stirred for 60 minutes at 30° C. The speed of the mechanical stirrer was adjusted to 700 rpm and allowed to stir for 60 minutes. The stirring was stopped and the plasticizer (PEG and/or PVP) was added to the flask. This mixture was stirred for 30 minutes under sonication at 700 rpm at 30° C. until a homogeneous, clear solution was obtained. The mechanical stirring was then stopped and the sonication was allowed to continue for an additional 30 minutes in order to remove all bubbles. The Erlenmeyer flask was then removed from the sonicator and left to sit at room temperature for 30 minutes. For the preparation of the films, a petri dish (150 mm diameter×15 mm height) was filled with about 150 g±2 g of the stock solution. The stock solution was subjected to different evaporation techniques. In a first experiment, a vacuum oven at 50° C. was used. The petri dishes were placed in the oven and the oven was evacuated using a vacuum pump. After 30 hours, the films obtained were yellow in color and some of the films exhibited curved surfaces. The experiments were repeated at 45° C., 40° C., and 35° C., under the same vacuum conditions. All of the experimental conditions above yielded colored films or films with non-uniform weight distribution. It was also observed that the higher the temperature, the darker and more intense the yellow color became. A preferred evaporation technique included evaporation in a chamber equipped with a variable-speed exhaust at room temperature. The airflow, temperature, and humidity were all measured during the evaporation process. The technique described above produced uniform evaporation and films with consistent thickness.

The film casting method can be used to produce exemplary polymeric eye inserts according to the present invention, e.g., for incorporation of pharmaceutically active agents, for example, 4-(7-Hydroxy-2-isopropyl-4-oxo-4H-quinazolin-3-yl)-benzonitrile.

In an embodiment, there is provided a method of preparing a polymeric insert according to the present invention, comprising preparing a mixture comprising the one or more mucoadhesive polymers, optionally, the plasticizer or softener, and one or more pharmaceutically active agent in a suitable first solvent. In additional or alternative embodiments, the mixture of the one or more mucoadhesive polymers, optionally, the plasticizer or softener, and one or more pharmaceutically active agent is stirred and/or sonicated. The one or more mucoadhesive polymers, the optional plasticizer or softener, and the one or more pharmaceutically active agent may be added to the solvent simultaneously or consecutively in any order. In some embodiments, the one or more mucoadhesive polymers and the optional plasticizer or softener are dissolved in the first solvent, optionally by stirring and/or sonication. In particular embodiments, the first solvent is water or other solvents in which the mucoadhesive polymers and the optional plasticizer or softener are soluble. In an embodiment, the pharmaceutically active agent may be added to the mixture either as a stock solution in a solvent or in neat form. In particular embodiments, the pharmaceutically active agent is dissolved in a second solvent that is miscible with the first solvent. In some embodiments, the first solvent is water and the second solvent is ethanol, acetone, or mixtures thereof. In a further embodiment, the mixture of the one or more mucoadhesive polymers, optionally, the plasticizer or softener, and one or more pharmaceutically active agents is evaporated to obtain a film, and optionally the film cut to obtain the drug containing polymeric insert. In a further embodiment, there is provided a polymeric eye insert obtainable by a method comprising the step of preparing a mixture comprising the one or more mucoadhesive polymers, optionally, the plasticizer or softener, and one or more pharmaceutically active agent in a suitable solvent, optionally with stirring and/or sonication. In some embodiments, the one or more mucoadhesive polymers and the optional plasticizer or softener are dissolved in the solvent, optionally by stirring and/or sonication. In particular embodiments, the solvent is water or other solvents in which the mucoadhesive polymers and the optional plasticizer or softener are soluble. In a further embodiment, the mixture of the one or more mucoadhesive polymers, optionally, the plasticizer or softener, and one or more pharmaceutically active agents is evaporated to obtain a film, and optionally the film cut to obtain the drug containing polymeric insert. In particular embodiments, the polymeric insert is sterilized by a suitable means, e.g., autoclaving or gamma radiation. Suitably, the pharmaceutically active agent is 4-(7-Hydroxy-2-isopropyl-4-oxo-4H-quinazolin-3-yl)-benzonitrile, lipoic acid choline ester salts. Suitably, the pharmaceutically active agent is 4-(7-Hydroxy-2-isopropyl-4-oxo-4H-quinazolin-3-yl)-benzonitrile. More suitably, the 4-(7-Hydroxy-2-isopropyl-4-oxo-4H-quinazolin-3-yl)-benzonitrile is present as crystal form B, characterized by an X-ray diffraction pattern having three or more peaks at 2θ values selected from 9.3° 10.6° 14.4°±0.2 °2 θ, typically, 9.3°, 10.6°, 14.4°, 15.5°, 17.9°, 19.9°, 23.4°±0.2 °2θ or more typically, 9.3, 10.6, 12.8, 14.4, 15.5, 17.9, 19.9, 21.3, 23.4, and 28.0±0.2 °2 θ. In some embodiments, the 4-(7-hydroxy-2-isopropyl-4-oxo-4H-quinazolin-3-yl)-benzonitrile is not dissolved in the solvent.

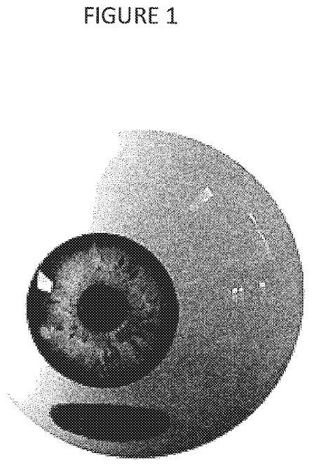

As discussed herein, in vivo studies indicate that traditional topical ophthalmic lubricants do not remain in the eye longer than approximately 25 minutes. However, use of one or more polymers combined with a plasticizer/softener, such as HP guar and hyaluronic acid blended with a plasticizer (such as PEG), may provide flexible films with tunable hydration and dissolution rates for comfortable insertion. While certain embodiments of the present invention are polymeric eye inserts containing a blend of hyaluronic acid, HP guar and PEG, it should be appreciated that other blends may be employed for polymeric eye inserts according to other embodiments of the present disclosure. FIG. 1 depicts placement of an eye insert according to an embodiment of the present invention on the surface of the eye.

In some embodiments of the present invention, the polymeric eye inserts described herein are a platform to deliver lubricants or other pharmaceutically active agents to treat ocular surface symptoms (such as redness, itching and dryness) and ocular diseases. In some embodiments, the polymeric eye inserts can be used to prolong exposure of pharmaceutically active agents or provide extended drug delivery of pharmaceutically active agents to the eye. Thus, in some embodiments, the present disclosure provides a method of providing extended drug delivery or prolonging exposure of a pharmaceutically active agent to the eye, by administering a polymeric eye insert including the pharmaceutically active agent to a patient in need thereof. In some embodiments, the exposure of the pharmaceutically active agent is prolonged by 1, 2, 3, 4, 5, or 6 hours when compared to an equivalent amount of pharmaceutically agent delivered in a solution or suspension formulation. In particular embodiments, the pharmaceutically active agent is 4-(7-hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone. In particular embodiments, the pharmaceutically active agent is 4-(7-hydroxy-2-isopropyl-4-oxo-4H-quinazolin-3-yl)-benzonitrile.

In some embodiments, the present invention relates to a polymeric eye insert, the insert comprising:

one or more mucoadhesive polymers that are biocompatible with the ocular surface and tear film of the eye; and
one or more pharmaceutically active agent, wherein a pharmacokinetic profile of the active agent in rabbit is achieved with at least a 5, 6, 7, 8, 9, or 10 fold lower dose of the active agent in the eye insert relative to the pharmacokinetic profile in rabbit studies of the active agent not formulated in an eye insert (e.g., in solution or suspension). In particular embodiments, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone. In particular embodiments, the pharmaceutically active agent is 4-(7-hydroxy-2-isopropyl-4-oxo-4H-quinazolin-3-yl)-benzonitrile.

In some embodiments of the present invention, the present disclosure provides a method of treating or reducing the symptoms of an ocular disease or disorder in a subject in need thereof, comprising administering a polymeric eye insert according to the present disclosure to the subject, wherein the subject suffers from one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, or episclentis. In particular embodiments, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone. In alternative embodiments, the pharmaceutically active agent is 4-(7-hydroxy-2-isopropyl-4-oxo-4H-quinazolin-3-yl)-benzonitrile.

In some embodiments of the present invention, the present disclosure provides a method of treating or reducing the signs and/or symptoms of dry eye disease (keratoconjunctivitis sicca), primary Sjogren's syndrome, or vernal keratoconjunctivitis, comprising administering a polymeric eye insert according to the present disclosure to a patient in need thereof. In particular embodiments, polymeric eye insert includes an effective amount of one or more pharmaceutically active agents. In particular embodiments, the pharmaceutically active agent is 4-(7-Hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-Methanesulfonyl-5-methyl-pyridin-3-yl)-3,4-dihydro-2H-benzo[1,4]oxazin-6-yloxy]-pyrrolidin-1-yl}-2-methoxy-ethanone, {(S)-3-[4-(6-Chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof. In particular embodiments, the pharmaceutically active agent is 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone. In alternative embodiments, the pharmaceutically active agent is 4-(7-hydroxy-2-isopropyl-4-oxo-4H-quinazolin-3-yl)-benzonitrile.

In some embodiments of the present invention, the present disclosure provides a method of treating or reducing eye pain in a subject in need thereof, by ocularly administering a polymeric eye insert according to the present disclosure to the subject. In particular embodiments, the polymeric eye insert includes an effective amount of a TRPV1 (transient receptor potential cation channel subfamily V member 1) inhibitor. In particular embodiments, the polymeric eye insert includes an effective amount of 4-(7-Hydroxy-2-isopropyl-4-oxo-4H-quinazolin-3-yl)-benzonitrile (compound I). In particular embodiments, the polymeric eye insert includes 4-(7-Hydroxy-2-isopropyl-4-oxo-4H-quinazolin-3-yl)-benzonitrile at a concentration of about 0.5% w/w, about 1.0% w/w, about 1.5% w/w, about 2.0% w/w, about 2.5% w/w, about 3.0% w/w, or about 3.5% w/w, based on the dry weight of the polymeric eye insert. In particular embodiments, the polymeric eye insert includes 4-(7-hydroxy-2-isopropyl-4-oxo-4H-quinazolin-3-yl)-benzonitrile or salts thereof in an amount of from about 0.01 mg to about 10 mg, from about 0.01 mg to about 5 mg, from about 0.01 mg to about 1 mg, from about 0.01 mg to about 0.5 mg, from about 0.01 mg to about 0.1 mg, from about 0.01 mg to about 0.08 mg per polymeric eye insert. In particular embodiments, the polymeric eye insert includes 4-(7-hydroxy-2-isopropyl-4-oxo-4H-quinazolin-3-yl)-benzonitrile or salts thereof in an amount of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.15 mg, about 0.2 mg, about 0.25 mg, about 0.3 mg, about 0.35 mg, about 0.4 mg, about 0.45 mg, or about 0.5 mg per polymeric eye insert.

In one embodiment, the present disclosure provides a method of treating or reducing ocular surface pain in a subject in need thereof, comprising ocularly administering a polymeric eye insert according to the present disclosure to the subject. In some embodiments, the ocular surface pain is episodic or acute pain. In some embodiments, the ocular surface pain is chronic ocular surface pain (COSP), lasting for at least about three months.

In some embodiments of the methods to treat or reduce ocular surface pain described herein, the ocular surface pain or chronic ocular surface pain is associated with dry eye disease. In some embodiments, the administration results in a decrease in the symptoms of dry eye disease. In particular embodiments, the administration results in a decrease in the ocular pain associated with dry eye disease. In some embodiments, the administration results in reduced incidence of at least about 10% in one or more of ocular dryness, ocular discomfort, ocular hyperemia, ocular burning or stinging, grittiness or foreign body sensation, or photophobia.

Dry eye disease is generally understood to be a complex, multifactorial condition characterized by inflammation of the ocular surface and lacrimal glands and reductions in the quality and/or quantity of tears. It is believed that up to 30% of dry eye disease patients suffer from ocular surface pain that may be chronic, i.e., lasting at least 12 weeks or three months. Thus, in some embodiments, the invention results in a decrease of at least about 10% in the symptoms of dry eye disease, including one or more of ocular dryness, ocular discomfort, ocular hyperemia, ocular burning or stinging, grittiness or foreign body sensation, or photophobia.

In some embodiments of the methods to treat or reduce ocular surface pain described herein, the subject suffers from one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, patients recovering from neurotrophic keratitis, or ocular pain persisting for at least three months after photorefractive keratectomy (PRK) surgery or laser-assisted in situ keratomileusis (LASIK) surgery.

In some embodiments of the methods to treat or reduce ocular surface pain described herein, the administration of a polymeric eye insert according to the present disclosure to the subject results in a reduction in a pain score of at least about 3, at least about 4, at least about 5, at least about 6, at least about 7, at least about 8, at least about 9 or at least about 10, compared to a placebo, when measured on a visual analog scale (VAS). In further embodiments, the administration of a polymeric eye insert according to the present disclosure to the subject results in a reduction in the subject's pain score of at least about 6, at least about 7, at least about 8, at least about 9 or at least about 10, compared to a placebo, when measured on the VAS.

In some embodiments of the methods to treat or reduce ocular surface pain described herein, the administration of a polymeric eye insert according to the present disclosure to the subject results in a reduction in the subject's pain of at least about 10%, at least about 15%, at least about 20%, or at least about 25%, compared to a placebo. In some embodiments, the reduction in the pain score arises from the difference in pain scores prior to and after administration of the polymeric insert to the subject. In some embodiments of the methods described herein, the reduction in pain score occurs within about half hour after administration of the polymeric insert to the subject.

In some embodiments of the methods to treat or reduce ocular surface pain described herein, the administration of a polymeric eye insert according to the present disclosure to the subject results in a reduction in hyperemia in the subject of at least about 1, at least about 2, at least about 3, at least about 4, or at least about 5, on the McMonnies scale.

In some embodiments of the methods to treat or reduce ocular surface pain described herein, the administration of a polymeric eye insert according to the present disclosure to the subject results in a reduction in pain score within about half hour, within about 1 hour, within about 2 hours, or within about 4 hours after administration of compound I to the subject.

In some embodiments of the methods described herein, the administration of a polymeric eye insert according to the present disclosure to the subject results in a reduction in hyperemia in the subject of at least about 1, at least about 2, at least about 3, at least about 4, or at least about 5, on the McMonnies scale.

In some embodiments of the methods described herein, the administration of a polymeric eye insert according to the present disclosure to the subject does not result in a change in one or more of best corrected visual acuity, intraocular pressure, slit-lamp biomicroscopy, dilated eye exam, blink rate, or tear production, compared to a placebo.

Thus, in some embodiments, the present invention relates to a method of treating or reducing ocular hyperemia in a subject in need thereof, comprising ocularly administering a polymeric eye insert according to the present disclosure to the subject. In some embodiments, the ocular hyperemia is associated with one or more of dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, or patients recovering from neurotrophic keratitis. In particular embodiments, the ocular hyperemia is associated with dry eye disease. In some embodiments of the methods described herein, the ocular hyperemia persists for at least three months after photorefractive keratectomy (PRK) surgery or laser-assisted in situ keratomileusis (LASIK) surgery.

In some embodiments, the invention relates to a method of treating dry eye disease in a subject in need thereof, comprising ocularly administering a polymeric eye insert according to the present disclosure to the subject.

In some embodiments, the methods described herein include administering an additional therapeutic agent to the subject. Further therapeutic agents may include, for instance, other compounds and antibodies useful for treating ocular surface disorders. A non-limiting list of such agents incudes nonsteroidal anti-inflammatory drugs such as ketorolac, nepafenac, bromfenac, corticosteroids; drugs for dry eye disease such as cyclosprine, lifitegrast, or other TRPV1 inhibitors. In particular embodiments, the additional therapeutic agent is an ophthalmic steroid such as dexamethasone, fluocinolone, loteprednol, difluprednate, fluorometholone, prednisolone, prednisone, medrysone, triamcinolone, betamethasone, rimexolone, or pharmaceutically acceptable salts thereof. Further non-limiting examples of such additional therapeutic agents that may be included in the pharmaceutical composition include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

All the aforementioned embodiments relating to the methods of treatment of certain ocular diseases are equally applicable to:

the polymeric eye insert for use in the treatment of the aforementioned ocular diseases according to the present invention (e.g., comprising a TRPV1 inhibitor, in particular 4-(7-hydroxy-2-isopropyl-4-oxo-4H-quinazolin-3-yl)-benzonitrile);

the use of the polymeric eye insert for the treatment of the aforementioned ocular diseases according to the present invention; and

a pharmaceutical composition in the form of a polymeric eye insert for use in the treatment of the aforementioned ocular diseases according to the present invention.

In some embodiments of the present invention, the present disclosure provides a method of providing a once a day delivery of a pharmaceutically active agent to a subject's eye by administering a polymeric eye insert comprising the pharmaceutically active agent to the subject. In some embodiments, the administration of the pharmaceutical active agent containing polymeric eye insert maintains a therapeutically effect amount of the pharmaceutically active agent in the eye for about 24 hours after administration. In some embodiments, the pharmaceutical active agent containing polymeric eye insert maintains a therapeutic effect after dissolution of the polymeric eye insert. In some embodiments, the present disclosure provides a method of providing a pharmaceutically active agent to a subject's eye by administering a polymeric eye insert comprising the pharmaceutically active agent to the subject once every two, three, four, five, six, or seven days.

The following non-limiting Examples are provided to illustrate embodiments of the invention.

### EXAMPLES

### Example 1

In an embodiment of the present disclosure, hyaluronate-fluorescein (Creative PegWorks; Chapel Hill, NC), sodium hyaluronate (Novozyme; Franklinton, NC), HP guar, HP guar-fluorescein, PEG 400, and water may be used to form a polymeric eye insert comprising HP guar and sodium hyaluronate; however, it should be appreciated that more or fewer components from different lots and/or distributors may be used to form a polymeric eye insert without departing from the present disclosure.

In order to form this HP guar/sodium hyaluronate insert, approximately 100 mL of water was added to an Erlenmeyer flask that had been autoclaved for approximately 30 minutes. The water was at a temperature of approximately 22 degrees Celsius. The HA component was tagged with fluorescein isothiocyanate (FITC) for tracking in vivo release. FITC-hyaluronic acid (approximately 102.2 mg) was then added to the water while stirring at approximately 23 degrees Celsius at a setting of 500 (1/min) using an IKA® Ret Control-Visc C hotplate/stirrer. Sodium hyaluronate (approximately 354.3 mg) was then added followed by HP guar (454.1 mg) and PEG 400 (approximately 97.2 mg). Additional water (approximately 100 mL) was then added. The mixture was stirred for approximately 20 hours at ambient temperature (approximately 22 degrees Celsius) using a stirring setting of 600 (1/min). The solution was then poured into a sterile polystyrene disposable petri dish (VWR, diameter of 55 mm, height of 15 mm). The petri dish containing the solution was then placed in a Lindberg Blue M convection oven (Thermo Scientific), heated at approximately 35 degrees Celsius, and then dried under high vacuum at approximately 23 degrees Celsius for approximately 1-2 days.

The resulting composition for this embodiment of a polymeric eye insert was as follows: 102.2 mg (approximately 10%) FITC-hyaluronic acid/354.3 mg (approximately 35%) sodium hyaluronate, 454.1 mg (approximately 45%) HP guar, and 97.2 mg (approximately 9%) PEG 400. Discs having a diameter of 6 mm were then punched out for in vivo assessment studies. While a methodology for forming an HP guar/hyaluronic acid insert according to an embodiment of the present disclosure has been described, it should be appreciated that other methodologies may be employed to form these or similar polymeric eye inserts without departing from the present disclosure.

An in vivo tolerability study was performed using single polymeric eye inserts and New Zealand white rabbits. The polymeric eye inserts utilized in this study were composed of 3-7 mm discs containing an HP guar/hyaluronic acid blend using PEG as a plasticizer. The hyaluronic acid component was tagged with fluorescein isothiocyanate (FITC) for tracking in vivo release. This study revealed acceptable tolerability and comfort using a 200 μm thick film with a diameter of 6 mm. An in vivo retention study also was performed using a single film of HP guar/hyaluronic acid/PEG blend (using 5% FITC-hyaluronic acid). The film hydrated in the cul-de-sac of the eye but fragments remained after two hours. However, these fragments may be explained through low frequency and intermittent blinking associated with the rabbit subjects. Results from measuring fluorescence of these polymeric eye inserts are shown in TABLE 1:

TABLE 1
FLUORESCENCE OF THIN FILM INSERTS-
TIME POINTS WITH ≥ 1.5 × BASELINE LEVEL

| | Minutes Post-Dose | | | | | | | |
|---|---|---|---|---|---|---|---|---|
| Film Sample | 2 | 10 | 20 | 30 | 45 | 60 | 90 | 120 |
| Film #1 | | X | X | X | X | X | X | |
| Film #2 | | | X | X | X | X | X | |
| Film #3 | | | | | X | X | | |

All of the polymeric eye inserts were very well tolerated with no physical reactions, discharge, squinting, or pawing. Once placed, the insert remained in the eye with very little movement until dissolved. Inserts dissolved during the first 30 minutes after insertion. After one hour, lubricant residue was visible on the corneal surface. After 6 hours, residue was no longer present. It was determined that the 6-mm size was the largest diameter that would fit in the cul-de-sac without infringement over the corneal-scleral limbus. At 7 mm in diameter, the insert crossed the limbus. However, other diameters of inserts may be employed without departing from the present disclosure.

Tests to study polymeric eye inserts according to embodiments of the present disclosure have been performed using a Spectralis HRA-OCT. This is a diagnostic device that integrates SD-OCT with the cSLO fundus imaging. The Anterior Segment Module provided through the Spectralis may allow for imaging of anterior segment structures. SD-OCT imaging is desirable because it does not require a tagged test article, it offers both visual and quantitative properties, it provides direct micrometer measurement of the tear film/polymeric eye insert, and it allows for acquisition of tear film height from four quadrants of the eye in seconds. Through this image, pooling of the polymeric insert in the lower tear meniscus can be viewed.

### Example 2

A variety of polymer inserts were prepared by film casting in order to assess the compatibility of the polymers to make clear and/or reasonably transparent insert films. The following polymer formulations were prepared and evaluated using various concentration ratios of each specified polymer: HA/PEG, HA/PVP/PEG, HA/PVP, HA/HP-Guar/PEG, HP-Guar/PVP/PEG, HA/HP-Guar/PVP/PEG, HA/HP-Guar/PAA/PEG, HA/HP-Guar/HPMC/PEG. A description of the characterization methods of the insert films is provided below.

### 1. Morphology

The surface morphology of the insert films was tested using the appropriate microscope. The texture and the transparency of the insert films were investigated and the observations were recorded. If the film surface was found to be clear and transparent it was noted. If undissolved particulate or haziness were observed this was also noted.

### 2. Thickness Uniformity

Four films were sampled and their thicknesses were evaluated by cutting insert disks with 6 mm diameters. The thicknesses of the disks were measured. The positions of the disk cutting were selected randomly, in the middle and near the edges of each film. The disks thicknesses were measured using a Mitutoyo digital caliper. The mean and the standard deviation of 12 disks were calculated for each film.

### 3. Weight Uniformity

In order to determine the weight uniformity, four different films were selected and 12 disks with 6 mm diameters were cut. The positions of the disks cut were selected randomly on the original films. The weight of each disk was determined using a high accuracy Sartorius balance. The weight of each individual disk was measured and the average weight of 12 disks was determined for each film. The standard deviation for the 12 data points was calculated and recorded.

### 4. Percentage Moisture Absorption

For the percentage moisture absorption test, four circular films with 150 mm diameters were prepared. From each film, four circular disks with 20 mm diameters were cut. The four disks were placed inside a chamber containing a 100 ml saturated solution of aluminum chloride. The chamber was closed tightly for 72 hours. During this time, the disk surface appearance stayed clear. The disks were carefully removed from the chamber and the weight of each disk was measured. The percentage moisture absorption of each disk was calculated according to the following formula:

% MA=((Final weight-initial weight)/initial weight)×100

The average percentage moisture absorption for 12 disks cut from four different films was recorded.

### 5. Percentage Moisture Loss

The same films produced for percentage moisture absorption were used for the percentage moisture loss measurements. Four disks above were placed in a desiccator containing anhydrous calcium chloride for 72 hours. The disks were then removed from the desiccator and their weights were determined. The percentage moisture loss for each film was calculated using the equation below:

% ML=((initial weight−final weight)/final weight)×100

The average moisture loss for the 12 disks cut from four different films was recorded.

### 6. Folding Endurance

Four large, circular films with diameters of 150 mm were prepared. Four square films with 4 cm×4 cm dimensions were prepared from each film. The film strips were repeatedly folded at the same place until the films broke or visibly cracked. The number of times the films could be folded at the same place without breaking gave the value of folding endurance. The data was collected and the average results were recorded. The average folding endurance of 16 square strips cut from the four different films was determined.

### 7. Dissolution Time and pH of the Solution

Four circular disks with 6 mm diameters were cut from the main circular films with 150 mm diameters. The films were placed in a vial with 2 ml of DI water and the time required for complete dissolution was recorded. The average dissolution time and the standard deviation for each group were recorded.

### 8. Tensile Strength, Modulus, Displacement, and Percent Elongation

Four film strips with 4 cm×2 cm dimensions were used for each data point measurement. All films were inspected for air bubbles and physical imperfections. The film strips were held between two clamps positioned at 3 cm distances during the measurement. The cell load used was 5 Kilograms. The strips were pulled by the top clamp at a rate of 10 cm/min. The average tensile strength, modulus, and percent elongation were measured and reported. Formulations used to prepare test films are provided below with the polymer compositions and testing data. The TABLE 2 results indicate that the presence of 5% PVP in the HA/PEG formulation improved the flexibility and elasticity of the film. As shown in TABLE 3, the presence of P30V PVP and 30% HP guar also provided a film of relatively good elasticity and flexibility. The presence of carbopol in the tested formulations resulted in brittle films as shown by the data in TABLE 4. 200 ppm of menthol led to faster dissolution rates and produced stiff films as shown in TABLE 5. The films with <200 ppm menthol (such as 150 ppm) were improved with similar modulus and 00 elongation to same films without menthol added.

TABLE 2
HA/PVP AND PEG COMBINATIONS

| | Dissolution | Surface | Modulus | Tear strength | % | |
|---|---|---|---|---|---|---|
| Formulation | time (min) | pH | (MPa) | (MPa) | elongation | Description |
| HA (45.4%): | 19 | 7.22 | 151.33 | 4.04 (±0.49) | 11.67 (±2.19) | Films are clear, |
| PVP (45.4%): | | | (±15) | | | transparent, |
| PEG (9.2%) | | | | | | uniform, and |
| | | | | | | bendable |
| HA (65%): | 26.5 | 7.25 | 155 (± | 5.51 (±0.69) | 15.63 (±0.6) | Films are clear, |
| PVP (25%): | | | 19.5) | | | transparent, |
| PEG (10%) | | | | | | uniform, and |
| | | | | | | bendable |
| HA (85%): | 27.4 | 7.3 | 159 (± | 4.7 (±0.28) | 21.04 (±5.6) | Films are clear, |
| PVP (5%): | | | 9.8) | | | transparent, |
| PEG (10%) | | | | | | uniform, and |
| | | | | | | bendable |
| HA (90%): | 30 | 7.2 | 190 (± | 6.4 (±0.39) | 13.5 (±4.9) | Films are clear, |
| PVP (10%) | | | 2.6) | | | transparent, |
| | | | | | | uniform, and |
| | | | | | | bendable |

TABLE 3
HA/HP-GUAR/PVP/PEG COMBINATIONS

| | Dis- | | | | | |
|---|---|---|---|---|---|---|
| | solution | | | Tear | | |
| | time | Surface | Modulus | strength | % | De- |
| Formulation | (min) | pH | (MPa) | (MPa) | elongation | scription |
| HA(22.5%): | 14 | 7.32 | 110 ± | 3.59 ± | 33.85 ± | Semi- |
| HP-Guar | | | 9.24 | 0.42 | 9.8 | trans- |
| (22.5%): | | | | | | parent |
| PVP(45%): | | | | | | to opaque |
| PEG (10%) | | | | | | films |
| HA (30%): | 33 | 7.21 | 147 ± | 5.33 ± | 38.75 ± | Semi- |
| HP-Guar | | | 3.61 | 0.5 | 9.0 | trans- |
| (30%): | | | | | | parent |
| PVP(30%): | | | | | | to opaque |
| PEG (10%) | | | | | | films |
| HA(40%): | 37 | 7.2 | 174 ± | 7.15 ± | 19.38 ± | Semi- |
| HPguar | | | 18 | 0.45 | 6.03 | trans- |
| (40%): | | | | | | parent |
| PVP(10%): | | | | | | to opaque |
| PEG (10%) | | | | | | films |
| HA(42.5%): | 53 | 7.23 | 160 ± | 5.66 ± | 18.33 ± | Semi- |
| HP-Guar | | | 18 | 0.75 | 3.21 | trans- |
| (42.5%): | | | | | | parent |
| PVP(5%): | | | | | | to opaque |
| PEG (10% | | | | | | films |
| HA(45%): | 52 | 7.2 | 258 ± | 8.99 ± | 20.42 ± | Semi- |
| HP-Guar | | | 42 | 1.25 | 3.15 | trans- |
| (45%): | | | | | | parent |
| PVP(10%) | | | | | | to opaque |
| | | | | | | films |

TABLE 4
HA/HP-GUAR/PAA/PEG COMBINATION

| | Dissolution | Surface | | | | |
|---|---|---|---|---|---|---|
| | time (min) | pH | | Tear | | |
| | (not | (not | Modulus | strength | % | |
| Formulation | measured) | measured) | (MPa) | (MPa) | elongation | Description |
| HA(22.5%): | | | 229 ± | 2.7 | 6.46 | Stiff films |
| HP-Guar(22.5%): | | | 13.3 | | | |
| PAA(45%): | | | | | | |
| PEG (10% | | | | | | |
| HA(30%): | | | 252 ± | 4.41 | 7.29 | Stiff films |
| HP-Guar(30%): | | | 13.89 | | | |
| PAA(30%): | | | | | | |
| PEG (10%) | | | | | | |
| HA(40%): | | | 273 ± | 6.82 | 6.5 | Stiff films |
| HP-Guar(40%): | | | 9.71 | | | |
| PAA(10%): | | | | | | |
| PEG (10%) | | | | | | |
| HA(42.5%): | | | 218 ± | 7.2 | 10.00 | Stiff films |
| HP-Guar(42.5%): | | | 10 | | | |
| PAA(5%): | | | | | | |
| PEG (10%) | | | | | | |
| HA(45%): | | | 190 ± | 9.19 | 18.13 | Stiff Films |
| HP-Guar(45%): | | | 3.61 | | | |
| PAA(10%) | | | | | | |

TABLE 5
HA/HP GUAR/PEG COMBINATION WITH 200 PPM MENTHOL

| | | | | Tear | | |
|---|---|---|---|---|---|---|
| | Dissolution | Surface | Modulus | strength | % | |
| Formulation | time (min) | pH | (MPa) | (MPa) | elongation | Description |
| HA(45.4%): | 66 | 7.15 | 189.35 | 10.56 | 16.67 | The films are |
| HP-Guar(45.4%): | | | | | | transparent |
| PEG(9.2%) | | | | | | |
| HA(45.4%): | 20 | 7.19 | 248.32 | 7.63 | 11.46 | The films are |
| HP-Guar(45.4%): | | | | | | opaque in the |
| PEG(9.2%): | | | | | | presence of |
| Menthol | | | | | | menthol |
| (200 ppm) | | | | | | |
| HA(45.4%): | 47 | 7.15 | 187.67 | 7.99 | 18.13 | The films are |
| HP-Guar(45.4%): | | | | | | opaque in the |
| PEG(9.2%): | | | | | | presence of |
| Menthol | | | | | | menthol |
| (100 ppm) | | | | | | |

HA/HP Guar/PEG Film Characterization

Based on the data generated for the wide range of film compositions it was determined that a preferred polymer composition contained 45.4% hyaluronic acid (HA): 45.4% hydroxypropyl guar (HP guar): 9.2% polyethylene glycol (PEG 400) (referred to as Formulation 2 below). This film was prepared as follows:

| Formulation 2 Film | | | |
|---|---|---|---|
| Composition | | Plasticizer | Solvent Media |
| Hyaluronic | Hydroxypropyl | Polyethylene | Distilled |
| acid | guar | glycol | water |
| 5.107 grams | 5.107 grams | 1.035 grams | 750 ml |

In a 1 L Erlenmeyer flask, 750 ml of distilled water was poured into the flask followed by the addition of Hyaluronic Acid (5.107 grams). The flask was then placed into the sonicator and attached to an overhead mechanical stirrer. The mixture was allowed to stir and sonicate 30 minutes (±10 minutes) at a speed of 700 rpm at 25° C. to 35° C. until a homogeneous, clear solution was obtained. Hydroxypropyl guar (5.107 grams) was then added to the flask. The flask was placed back into the sonicator and attached to an overhead mechanical stirrer. The mixture was stirred and sonicated for 120 minutes (±10 minutes) at a speed of 700 rpm at 38° C. to 42° C. until a homogeneous, clear solution was obtained. The plasticizer, polyethylene glycol-400 (1.035 grams), was added into the flask. The mixture was allowed to stir and sonicate for 30 minutes (±10 minutes) at a speed of 700 rpm at 40° C. to 45° C. until a homogeneous, clear solution was obtained. The mixture was sonicated without stirring for an additional 30 minutes (±10 minutes) at 40° C. to 45° C. until a homogeneous, clear solution (no bubbles) was obtained. The flask was allowed to stand at room temperature for 30 minutes (±10 minutes). After proper mixing, the casting solution (150 g±2 g) was poured into a clean petri dish (150 mm×15 mm). The petri dish was dried at room temperature for 60 h (±5 h) in an evaporation chamber equipped with an exhaust fan. After drying, the disk was cut into 9 cm×9 cm pieces and kept in an airtight bag for 24 h (+3 h) under controlled humidity (<50%) and temperature (23° C. to 26° C.) levels for use in further characterization studies.

TABLE 7
FORMULATION 2 AVERAGE
WEIGHT MEASUREMENTS

| | Weight uniformity |
|---|---|
| | (mean ± SD) |
| Formulation-2 | mg |
| F-2-50-2 | 4.425 ± 0.263 |
| F-2-50-3 | 4.258 ± 0.188 |
| F-2-50-4 | 4.492 ± 0.156 |
| F-2-50-5 | 4.575 ± 0.280 |

TABLE 8
FORMULATION 2 MOISTURE
ABSORPTION MEASUREMENTS

| | % Moisture Absorption |
|---|---|
| Formulation-2 | (mean ± SD) |
| F-2-50-2 | 5.052 ± 0.211 |
| F-2-50-3 | 5.550 ± 0.289 |
| F-2-50-4 | 4.770 ± 0.327 |
| F-2-50-5 | 4.845 ± 0.606 |

TABLE 9
FORMULATION 2 MOISTURE
LOSS MEASUREMENTS

| | % Moisture loss |
|---|---|
| Formulation-2 | (mean ± SD) |
| F-2-50-2 | 8.630 ± 0.433 |
| F-2-50-3 | 9.010 ± 0.608 |
| F-2-50-4 | 9.148 ± 0.515 |
| F-2-50-5 | 8.415 ± 0.323 |

TABLE 10
FORMULATION 2 FOLDING
ENDURANCE MEASUREMENTS

| | Folding endurance |
|---|---|
| Formulation-2 | (mean ± SD) |
| F-2-50-2 | 38.50 ± 4.950 |
| F-2-50-3 | 42.50 ± 6.363 |
| F-2-50-4 | 37.50 ± 3.535 |
| F-2-50-5 | 36.50 ± 6.369 |

TABLE 11
FORMULATION 2 DISSOLUTION
TIME AND PH MEASUREMENTS

| Formu- | Dissolution time (min*) | | | pH | | |
|---|---|---|---|---|---|---|
| lation-2 | | Mean | SD | | Mean | SD |
| F-2-50-2 | 17 | 16.25 | 0.957 | 7.19 | 7.023 | 0.210 |
| F-2-50-3 | 17 | | | 7.16 | | |
| F-2-50-4 | 16 | | | 7.01 | | |
| F-2-50-5 | 15 | | | 6.73 | | |

TABLE 12
FORMULATION 2 TENSILE STRENGTH, MODULUS,
AND PERCENT ELONGATION MEASUREMENTS

| | Tensile | | | |
|---|---|---|---|---|
| | strength | Modulus | Displacement | % |
| | (N/cm2) | (N/cm2) | (from 3 cm) | Elongation |
| Formulation-2 | 9.8 | 9.9 | 4.1 | 36.66 |
| | 10.10 | 9.9 | 3.9 | 30 |
| | 9.90 | 10.3 | 3.9 | 30 |
| | 10.10 | 10.2 | 4.2 | 40 |
| Mean | 9.975 | 10.075 | 4.025 | 34.17 |
| SD | 0.15 | 0.206 | 0.150 | 5.0 |

### Example 3

Direct thickness measurement of the tear film is possible using HRA-OCT. HRA-OCT imaging was used to provide a measurement of the tear film thickness following insertion of the insert and this indirectly indicates the effect resulting from the delivery of lubricant (i.e., enhancement of the tear film thickness indicates delivery of lubricant and/or drug). Following insertion the insert is expected to slowly dissolve and release lubricant and/or drug. The general method used is described below using New Zealand rabbits. In this procedure, an insert using 45.4% Hydroxypropyl guar (HP guar) and 9.2% Polyethylene glycol (PEG 400) was evaluated in rabbits using HRA-OCT. On Day 1, a single insert was placed into the central, lower cul-de-sac of the right eye with forceps or another appropriate device. Treatment was repeated on Day 3 with inserts applied to the left eye. The study treatment design is summarized in TABLE 13.

TABLE 13
EXAMPLE STUDY DESIGN

| Group | Animal | | | Observation |
|---|---|---|---|---|
| No. | No. | Treatment* | Dosing Regimen | Period |
| 1 | 3 | TA1 | One (1) insert on | Day 3 after |
| 2 | 3 | TA2 | Day 1 in the right | the last |
| 3 | 3 | TA3 | eye | ophthalmic |
| | | | One (1) insert on | evaluation |
| | | | Day 3 in the left | |
| | | | eye. | |

Animals undergo optical coherence tomography (OCT) scans at various time points up to 3 hours if needed. The method for OCT imaging and image analysis in the rabbit is as follows:
1. Lighting was dimmed in the imaging room to facilitate imaging.
2. Lightly brush below the eye to be imaged with a cotton-tipped applicator to induce a natural blink response.
3. Capture one horizontal image centered at the apex of the cornea.
4. Lightly brush below the eye again with a cotton-tipped applicator to induce a natural blink response.
5. Capture one vertical image centered at the apex of the cornea.
6. Document dose information and image numbers.
7. Determine 3 points on each image for analysis (horizontal: nasal, apex, temporal region of the eye; vertical: top, apex, bottom of the eye).
8. Use the measurement tool on the Bioptogen to determine tear film thickness at each analysis point and document measurements.

Treatment groups and imaging schedules for the test animals are presented below in TABLES 14 and 15.

TABLE 14
TREATMENT GROUPS

| Group | | Hydration Drop | No. of |
|---|---|---|---|
| No. | Treatment | (BSS) | Animals |
| 1 | BSS | 30 uL dose every | 4 |
| | | 15 minutes | |
| 2 | 45.42% Sodium- | 30 uL dose every | 4 |
| | HA/45.42% HP- | 15 minutes | |
| | Guar/9.16% PEG400 | | |
| | Insert | | |
| 3 | 45.42% Sodium- | 30 uL dose at | 4 |
| | HA/45.42% HP- | insertion | |
| | Guar/9.16% PEG400 | | |
| | Insert | | |
| 4 | Lacrisert | N/A | 4 |

TABLE 15
GROUP IMAGING SCHEDULE

| Group | Imaging Schedule |
|---|---|
| 1 | Pre-dose |
| | Immediately Post-Dose |
| | 15 and 30 Min Post 1^{st} Dose |
| 2 | Pre-dose |
| | Immediately Post-Dose |
| | 15, 30, 45, 60, 75 & 90 Min Post-Dose |
| 3 | Pre-dose |
| | Immediately Post-Dose |
| | 15, 30, 45, 60, 75, 90, 105, 120, 135 & |
| | 150 Min Post-Dose |
| 4 | Pre-dose |
| | Immediately Post-Dose |
| | 15, 30, 45, 60, 75, 90, 105, 180 & 360 |
| | Min Post-Dose |

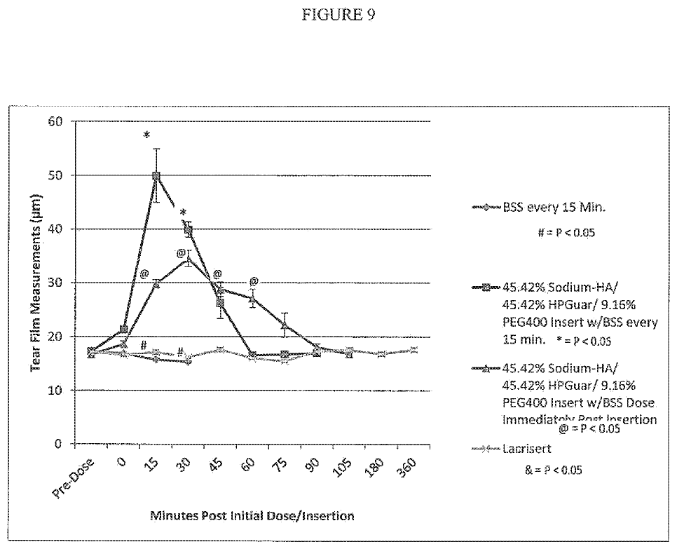

FIG. 9 presents the tear film thickness data from the testing. In this test LACRISERT® was used as a control. In this in vivo experiment there were no significant issues associated with safety or tolerability with LACRISERT® and the test articles containing HPGuar/HA/PEG. In this in vivo test the test articles were exposed to two different post-dosing regimens. In one case following insertion the BSS was added every 15 min to try and accelerate the dissolving insert. In the second case BSS was dosed once following the insert insertion. The LACRISERT® was simply inserted as per instructions for the human eye. The OCT measurements showed an increase in tear film thickness for the test articles for both scenarios. The BSS addition accelerated the dissolving insert as showed by the rapid increase in tear film thickness around 5 min to a max tear film thickness of 50 microns after 15 min. Comparatively, the scenario with a single post insertion drop showed the tear film thickness to extend across 90 min followed by decrease to baseline within 2 hours. The LACRISERT® during this time frame showed no noticeable effects on the tear film thickness and following 3 hours it remained in a solid-like state. The HA/HPG/PEG insert test articles were completely dissolved after 2 hours in this experiment.

### Example 4—Inserts with Pharmaceutically Active Agents

In specific aspects, the inserts of the present invention may include one or more pharmaceutically active agents, for example, as detailed herein. A non-limiting example of an insert film prepared with an anti-muscarinic, atropine, is provided below.

Eye Insert Preparation with Atropine

40% Hyaluronic acid (HA): 40% hydroxypropyl guar (HP): 10% Polyethylene glycol (PEG 400): 10% Polyvinyl pyrrolidone: 500 ppm Atropine

TABLE 17
ATROPINE INSERT FORMULATION

| Film formulation | | | Plasticizer | | Solvent |
|---|---|---|---|---|---|
| | Hydroxy- | Polyvinyl | Polyethylene | Drug | Media |
| Hyaluronic | propyl | pyrrolidone | glycol | Atropine | Distilled |
| acid | guar | (Aldrich) | (Aldrich) | (Aldrich) | water |
| 2.1 g | 2.1 g | 0.525 g | 0.525 gm | 0.175 g | 350 ml |

### Procedure:

To prepare 350 g of the insert formulation the following quantities are needed: HA (2.1 g): HP-guar (2.1 g): PEG-400 (0.525 g): PVP (0.525 g): Atropine (0.175 g) in 350 ml distilled water. In 1 L Erlenmeyer flask, 350 ml distilled water was mixed with 2.1 g Hyaluronic acid and 0.525 g polyvinyl pyrrolidone. The flask was attached to an overhead mechanical stirrer and the mixture was stirred at 600 RPM for 30 minutes at 35° C. Then 2.1 g Hydroxypropyl guar was added. The mixture was then stirred for 120 minutes at 38° C. until a homogeneous clear solution is obtained. The plasticizer polyethylene glycol-400 (0.525 g) and Atropine (0.175 g) were then added into the flask and the mixture was stirred for another 30 minutes at 700 RPM. The mixture was left to cool down for 30 minutes. At this stage the solution was ready for film casting.

### Film Casting:

150 g±2 g of the solution was poured into clean petri dish (150 mm×15 mm). The petri dish was dried for 30 h at room temperature for 30 h using a drying chamber. The obtained film was clear and did not show any crystallization or unusual visual appearance.

Eye Insert Preparation with Povidone Iodine

In another example, a broad spectrum biocide povidone iodine was utilized with the insert. This insert had the following formulation: 40% Hyaluronic acid (HA): 40% hydroxypropyl guar (HP): 10% Polyethylene glycol (PEG 400): 10% Polyvinyl pyrrolidone and 500 ppm PVP-I in the total mass.

Procedure:

Procedure for preparing 350 g batch of the formulation in 1 L Erlenmeyer flask with concentration 0.015 g/mL. HA (2.1 g): HP-guar (2.1 g): PEG-400 (0.525 g): PVP (0.525 g): PVP-I (0.175 g) in 350 ml distilled water. In 1 L Erlenmeyer flask, 350 mL distilled water, Hyaluronic acid (2.1 g) and polyvinyl pyrrolidone (0.525 g) was added. The flask was placed into the sonication bath and attached to overhead mechanical stirrer. The mixture was stirred and sonicated at the same time for 30 minutes (±10 minute) at a speed of 600 RPM and at a temperature between 25° C. to 35° C. until a homogeneous clear solution was obtained. The Hydroxypropyl guar (2.1 g) was then added. The flask content was stirred for 120 minutes (±10 minute) at a speed of 600 RPM and at a temperature between 38° C. to 41° C. until a homogeneous clear solution was obtained. The polyethylene glycol-400 (0.525 g) and PVP-I (0.175 g) were then added into the flask. The mixture was stirred for extra 45 minutes. 150 gm±2 g of the solution was poured in a clean petri dish (150 mm×15 mm). The petri dish was dried at room temperature for 30 h (±1 h) in a ventilated chamber. 500 ppm of PVP-I was calculated based on the total mass including water.

### Example 5

Tear film measurements for polymeric eye inserts according to embodiments of the present disclosure were also compared to tear film measurements of SYSTANE® ULTRA eye drops as well as GENTEAL® gel eye drops and PROVISC® injectable.

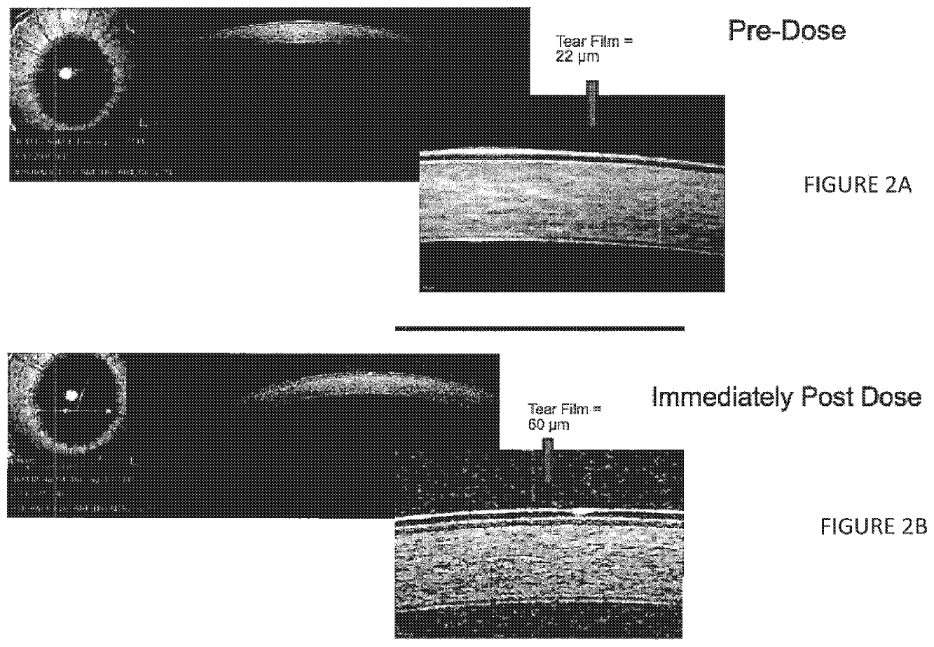

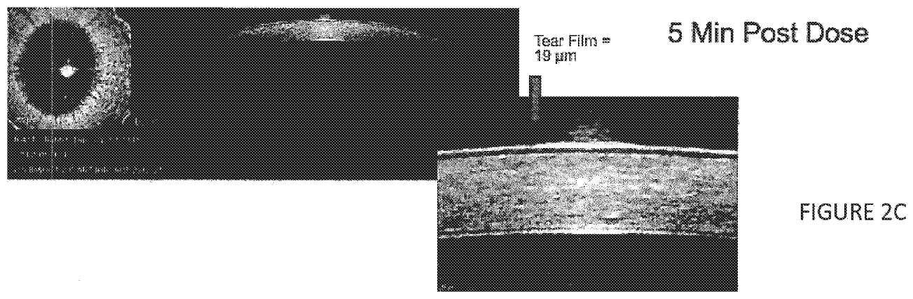

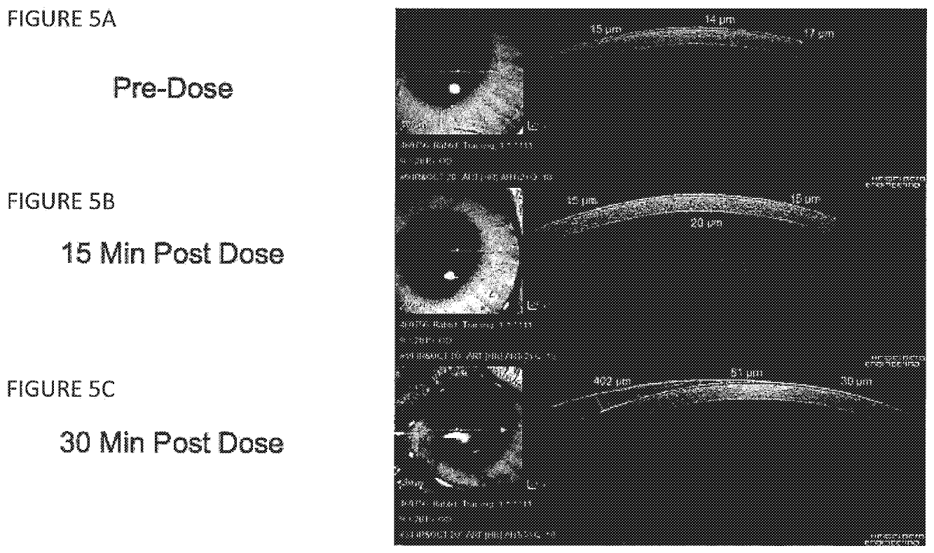

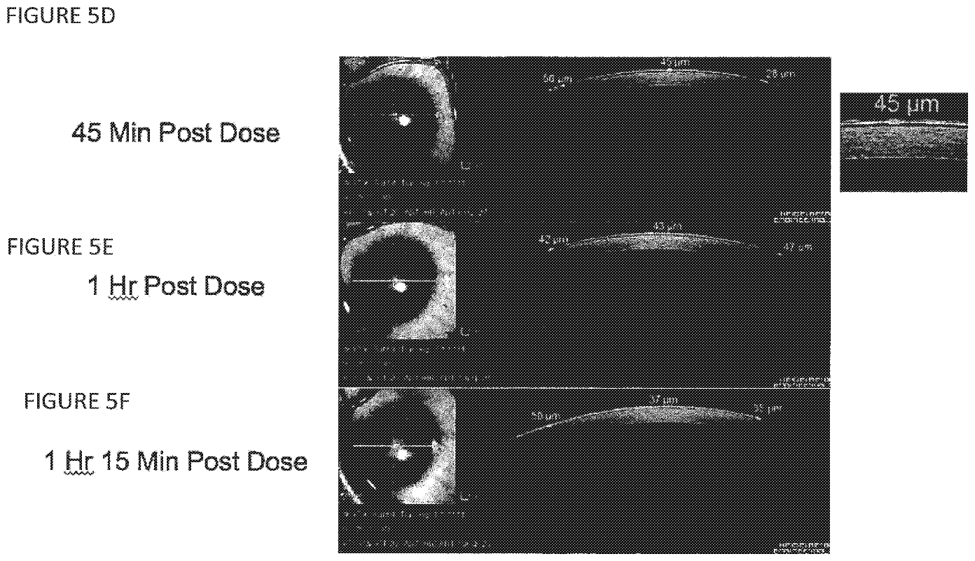

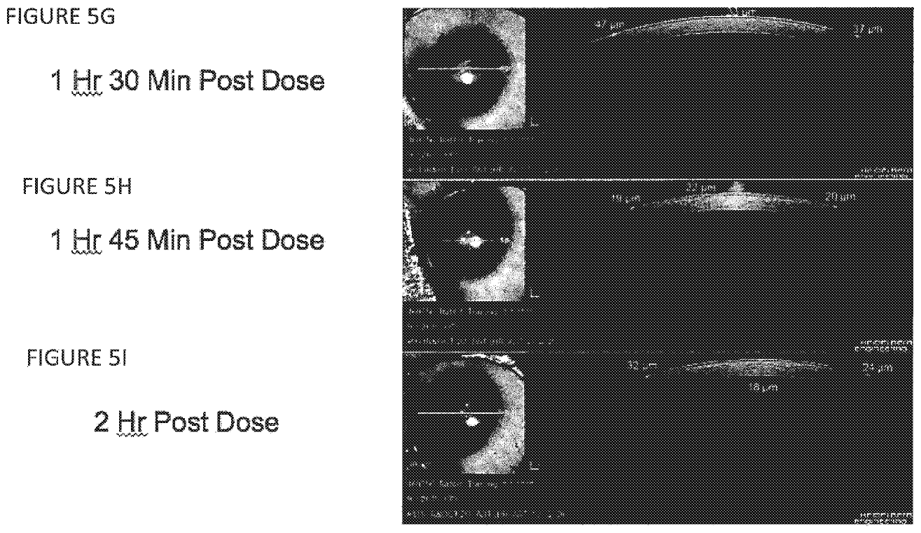

FIGS. 2A-2C depict tear film measurements for the SYSTANE® ULTRA eye drops pre-dose (FIG. 2A), immediately post-dose (FIG. 2B) and 5 minutes post-dose (FIG. 2C). FIGS. 2A-2C reflect that the tear film measures 22 μm pre-dose, 60 μm immediately post-dose, and 19 μm 5 minutes post-dose.

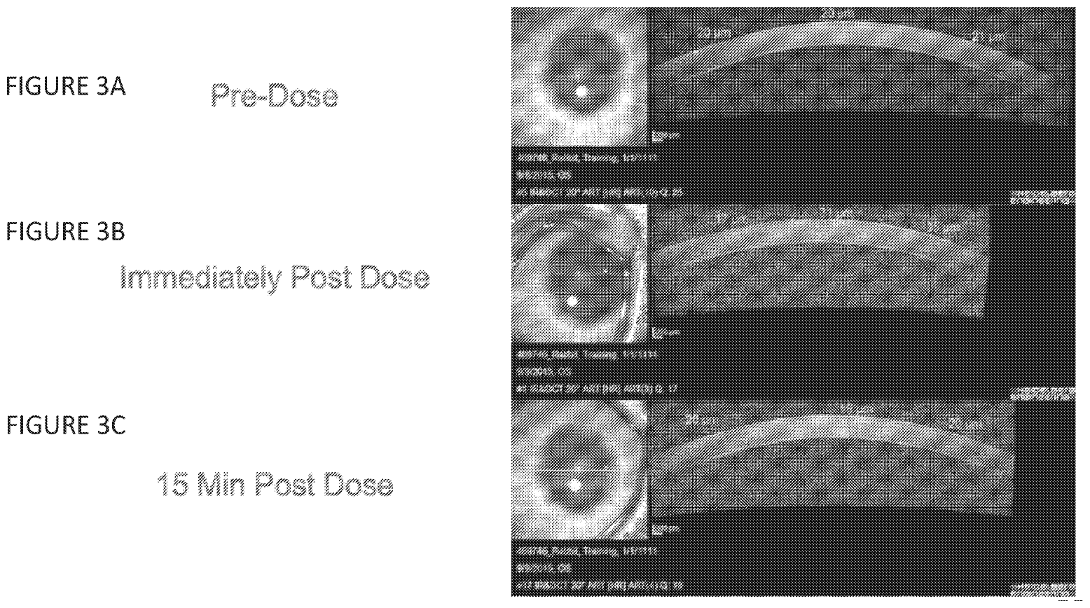

FIGS. 3A-3C depict tear film measurements for the GENTEAL® gel eye drops pre-dose (FIG. 3A), immediately post-dose (FIG. 3B) and 5 minutes post-dose (FIG. 3C). FIGS. 3A-3C reflect that the tear film measures 20 μm pre-dose, 31 μm immediately post-dose, and 19 μm 5 minutes post-dose.

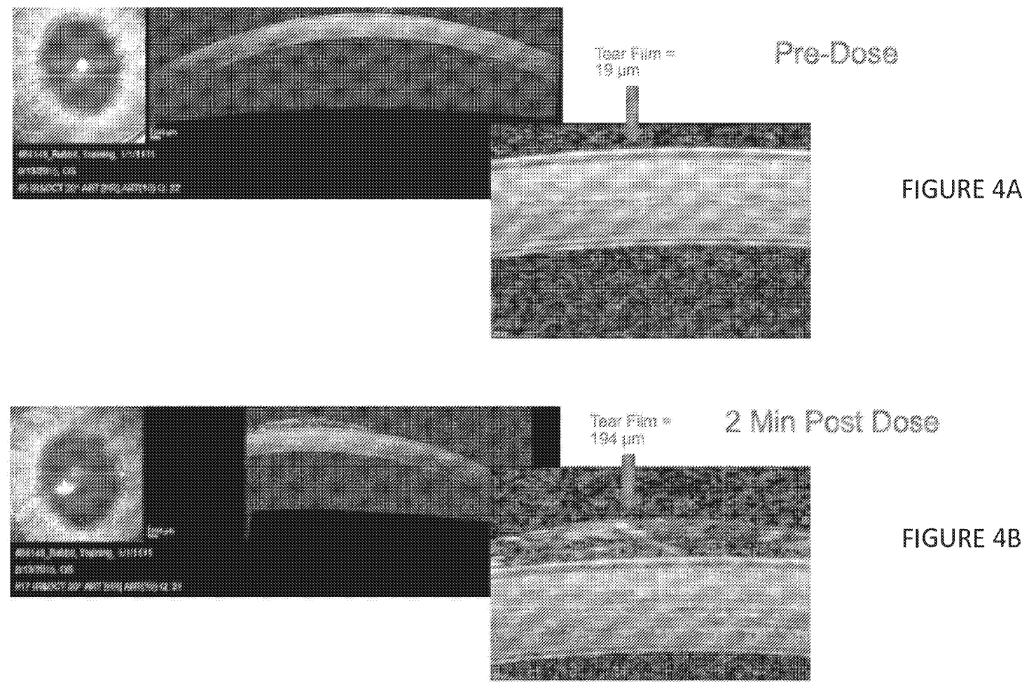

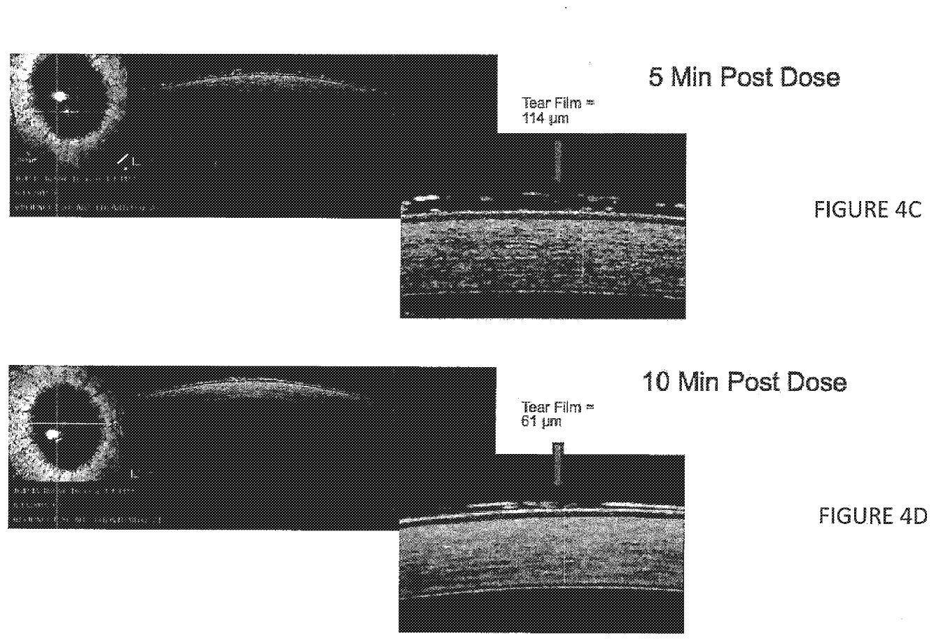

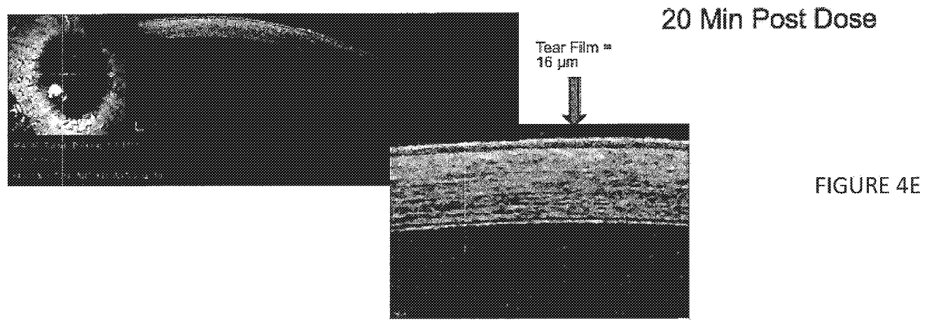

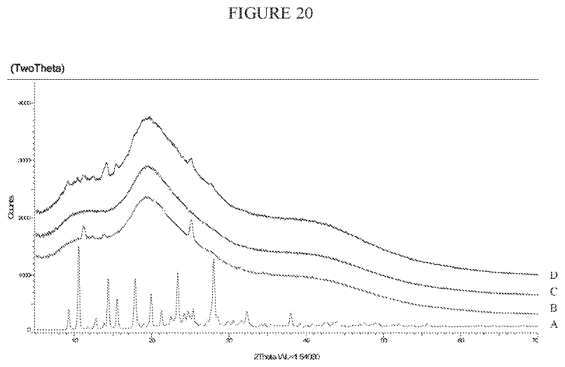

FIGS. 4A-4E depict tear film measurements for the PROVISC® injectable pre-dose (FIG. 4A), immediately post-dose (FIG. 4B), 5 minutes post-dose (FIG. 4C), 10 minutes post-dose (FIG. 4D) and 20 minutes post-dose (FIG. 4E). FIGS. 4A-4E reflect that the tear film measures 19 μm at pre-dose, 194 μm immediately post-dose, 114 μm at 5 minutes post-dose, 61 μm at 10 minutes post-dose, and 16 μm at 20 minutes post-dose.

Each of the tear film measurements set forth in FIGS. 2A-2C, 3A-3C, and 4A-4E reflect the tear film increases in thickness immediately post-dose but returns to a thickness similar to that measured pre-dose within anywhere from 5 to 20 minutes post-dose.

In contrast, FIGS. 5A-5I reflect tear film measurements associated with insertion of a polymeric eye insert according to embodiments of the present disclosure. These measurements reflect that the tear film measures 14 μm pre-dose (FIG. 5A), 20 μm 15 minutes post-dose (FIG. 5B), 81 μm 30 minutes post-dose (FIG. 5C), 45 μm 45 minutes post-dose (FIG. 5D), 43 μm 1 hour post-dose (FIG. 5E), 37 μm 1 hour and 15 minutes post-dose (FIG. 5F), 33 μm 1 hour and 30 minutes post-dose (FIG. 5G), 22 μm 1 hour and 45 minutes post-dose (FIG. 5H), and 18 μm 2 hours post-dose (FIG. 5I). Accordingly, in this embodiment of the present disclosure, the tear film thickness does not return to its pre-dose thickness until approximately 2 hours post-dose.

Additional tear film measurements were performed on New Zealand white rabbits. Each rabbit received a single polymeric eye insert. 3 horizontal and 3 vertical images were obtained per time point. Three points on each line were measured and zoomed in to 800% to determine the depth of the tear film/test article.

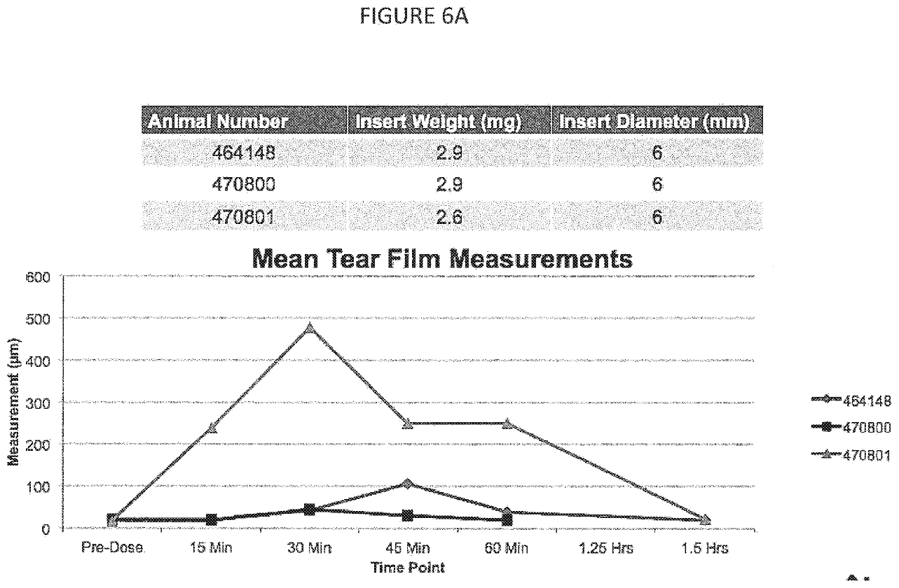

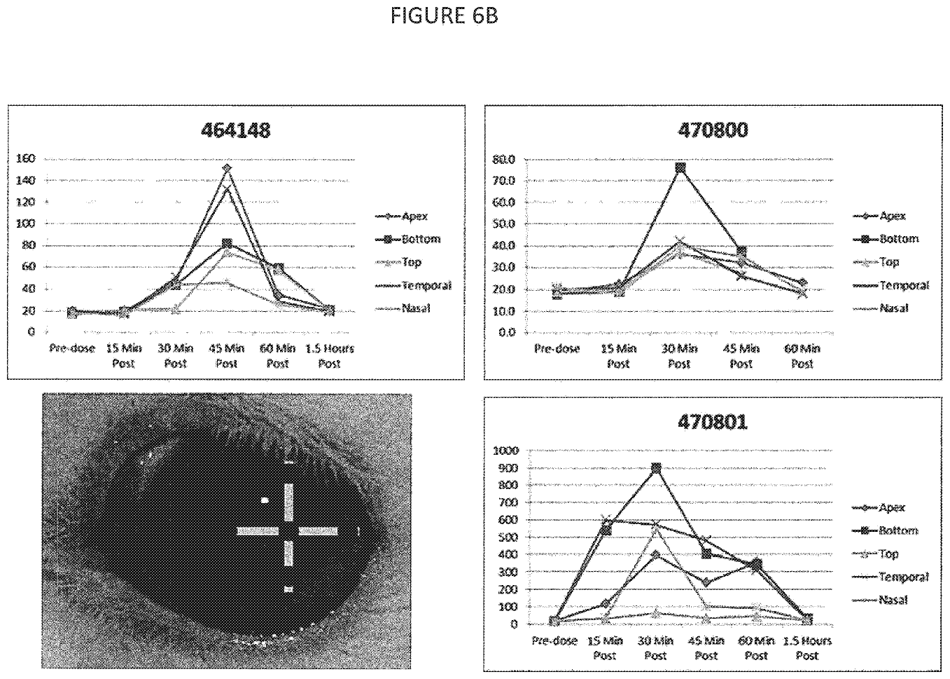

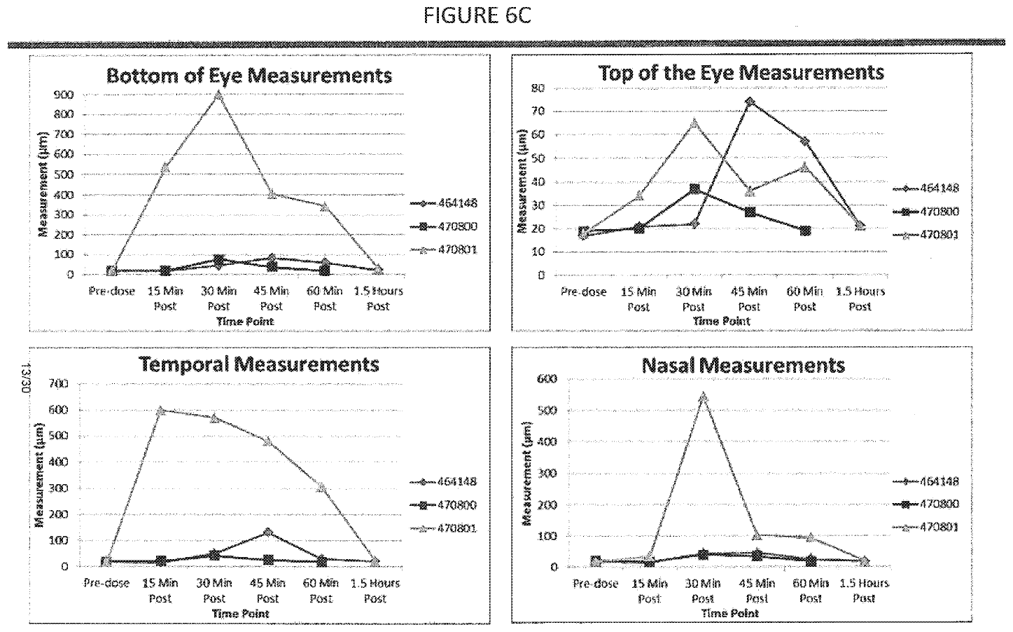

FIG. 6A reflects mean tear film measurements using polymeric eye inserts according to embodiments of the present disclosure. Three rabbits were tested, and each rabbit blinked three times prior to image capture. The insert diameter (6 mm) remained the same across testing of each rabbit, and the insert weight ranged from 2.6 mg to 2.9 mg. FIG. 6B reflects tear film measurements by individual animal. FIG. 6C reflects tear film measurements based on location in the eye including bottom of the eye, top of the eye, temporal and nasal measurements.

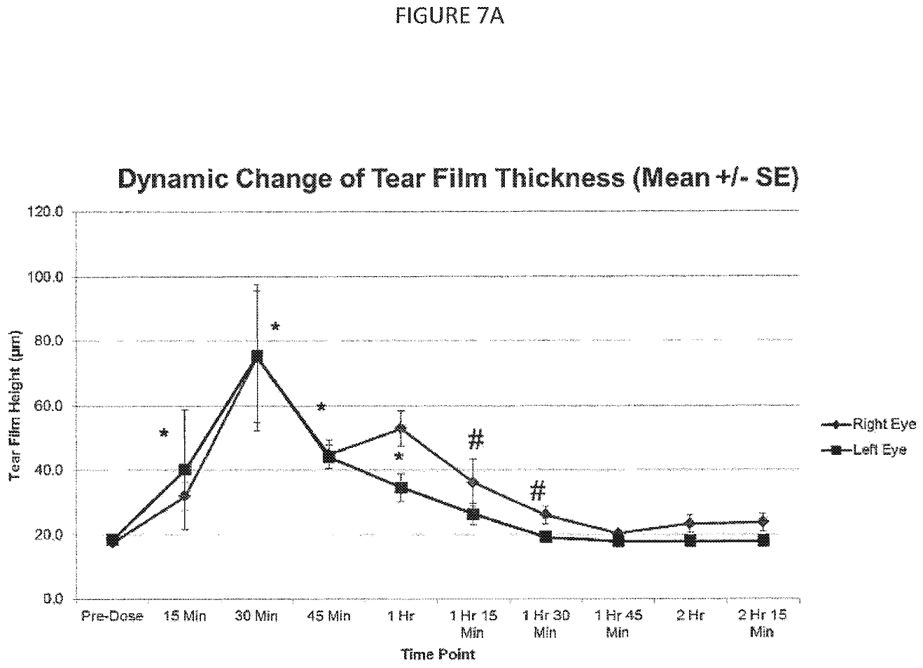

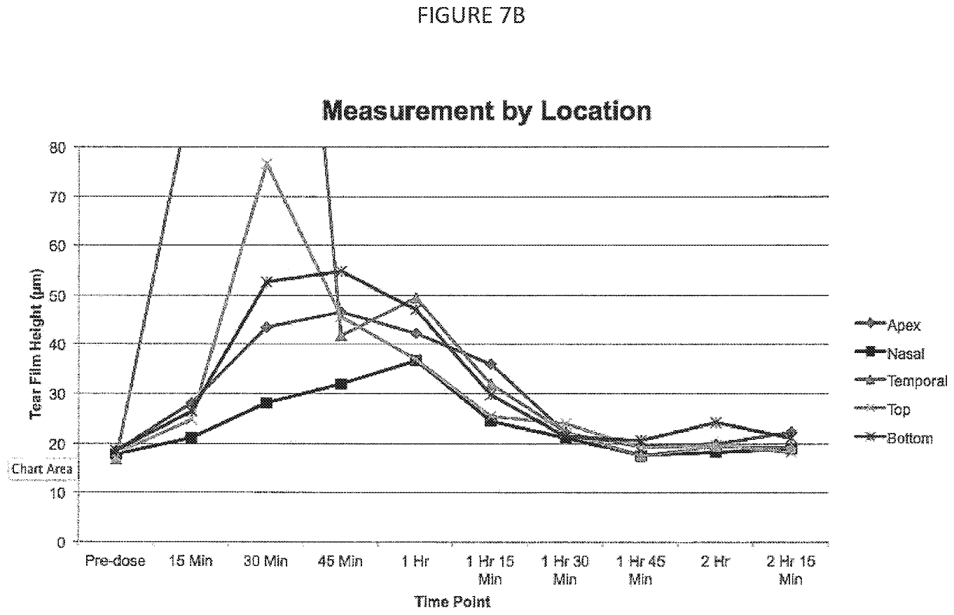

Further testing on New Zealand white rabbits measured the dynamic change of tear film thickness associated with polymeric eye inserts according to embodiments of the present disclosure (FIG. 7A). The insert diameter remained at 6 mm. The insert weight for oculus sinister (OS) ranged from 3.2 to 3.8 mg, and the insert weight for oculus dextrus ranged from 2.2 to 2.6 mm. FIG. 7B reflects tear film measurements by location (apex, nasal, temporal, top, and bottom).

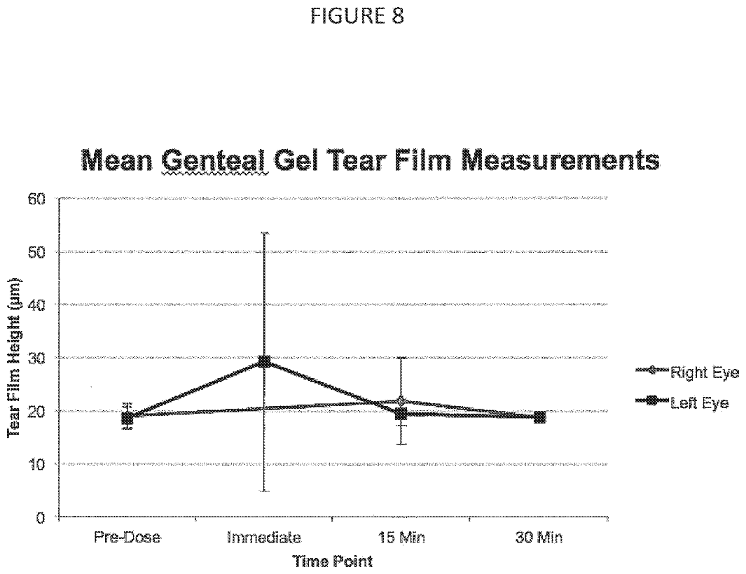

This testing also made similar measurements with respect to GENTEAL® gel at a dosage of 80 μL or approximately 76.3 mg into the central lower cul-de-sac of the eye. FIG. 8 reflects mean GENTEAL® gel tear film measurements for the right and left eye.

After taking measurements for both the polymeric eye insert according to embodiments of the present disclosure and GENTEAL® gel, the results were analyzed. TABLE 18 below reflects the number of animals with a mean of 6 readings greater than or equal to 30 μm.

TABLE 18

| | Minutes Post Dose | | | | | | | | |
|---|---|---|---|---|---|---|---|---|---|
| Test | 15 | 30 | 45 | 60 | 75 | 90 | 105 | 120 | 135 |
| Article | | | | | | | | | |
| Genteal | 1 | 0 | 0 | 0 | 0 | 0 | 0 | 0 | 0 |
| Gel | | | | | | | | | |
| (microns) | | | | | | | | | |
| Eye | 3 | 6 | 7 | 5 | 2 | 1 | 0 | 1 | 1 |
| Insert | | | | | | | | | |
| (microns) | | | | | | | | | |

This testing confirmed that tear film thickness change can be effectively monitored through Spectralis HRA+OCT. Inserts begin to dissolve by 15 minutes post-dose in most animals. Most animals dosed with a polymeric eye insert according to embodiments of the present disclosure had a significant increase of tear film thickness for at least 30 minutes post-dose. It should be appreciated that the location of the polymeric eye insert, both initial placement and movement after blinking, may create variations in data, particularly in early time points; however, IR image and OCT can help to differentiate the influence of insert location. It also should be appreciated that the weight of the insert may have an impact on length of retention. Further, it should be appreciated that intrinsic differences among animals may impact the results. For example, one animal had the longest duration of increased tear film thickness regardless of insert size; however, the larger insert retained approximately 45 minutes longer. It also was noted that aqueous solutions caused little tear film thickness changes.

As reflected through the studies described above, a polymeric eye insert according to an embodiment of the present disclosure may assume the form of a dissolvable film comprised of hydrophilic polymers with high mucoadhesive and H-bonding properties. The film may contain one or more naturally derived polysaccharides or synthetic polymers that are biocompatible and well-tolerated by the eye. The dissolvable film may have a thin film design that may allow for easy, comfortable insertion into the cul-de-sac of the eye, as the film should be small enough to fit into the cul-de-sac with little-to-no irritation upon insertion but large enough so that dissolution occurs over a longer period of time. Such a dissolvable film may hydrate quickly to form a soluble gel and release lubricant and/or a pharmaceutically active agent within a short time frame (e.g., the first 5-10 minutes following insertion). This slow pulsed flow of lubricant may maximize the adhesion and residence time of the lubricant on the ocular surface as compared to topical drop usage. The retention time of the lubricant on the eye may be increased by slow delivery in the tear film and ocular surface.

Insertion of a dissolvable film according to embodiments of the present disclosure does not lead to visual disturbances after several minutes. It should be appreciated that the dissolvable film may retain a lubricant for approximately two hours or more; however, there may be embodiments of the present disclosure where retention may occur over approximately 30-60 minutes. Accordingly, a dissolvable film or polymeric eye insert according to embodiments of the present disclosure may provide advantages, including but not limited to, quick dissolution for reduced blurring, a thin film design for enhanced wetting kinetics and ocular tolerability, improved comfort on insertion, and reduced foreign sensation. Further, tolerability and delivery of lubricant may be improved as compared to other topical delivery systems or inserts.

While embodiments of the present disclosure have been described for use as lubricants and/or pharmaceutically active agents to treat dry eye, it should be appreciated that polymeric eye inserts according to embodiments of the present disclosure also may have advantages for ophthalmic delivery of pharmaceutically active agents to treat other ocular disorders. A non-exhaustive list of such disorders includes ocular hypertension, glaucoma, glaucomatous retinopathy, optic neuropathy, macular degeneration, diabetic retinopathy, choroidal neovascularization, proliferative vitreoretinopathy, ocular wounds and infections, presbyopia, and myopia.

While some embodiments have been described as films, it should be appreciated that a polymeric eye insert according to embodiments of the present disclosure can assume a variety of shapes including, but not limited to, films, rods and spheres. In an embodiment of the present disclosure, a circular film of approximately 0.5 to 10 mm diameter may be employed. In other embodiments, circular films of 4-7 mm diameter are particularly preferred. Various other film shapes may be used in certain embodiments, such as those presented in FIGS. 10A-10C.

Regardless which shape the insert assumes, a polymeric eye insert according to embodiments of the present disclosure should be small enough to fit into the cul-de-sac of the eye and be rapidly wetted so that there is little or no irritation upon insertion. The insert also should be large enough to allow for dissolution over anywhere from approximately 30-120 minutes to allow for release of the lubricant(s) and/or pharmaceutically active agents to occur. The insert should also have a thickness that is relatively comfortable for the user. A preferred thickness is between 50-250 microns, and a most preferred thickness is between 70-150 microns. The target thickness is 90 microns for films dissolving in less than 2 hours.

### Example 6

Monkey Tolerability Study

The cynomolgus male monkey of Chinese origin (protein-naïve) was selected for this study based on the pharmacological and anatomical relevance of the monkey eye and following tolerance assessment in rabbit. The monkey eye blinks with similar frequency to the human. Clinical observations were performed for tolerability of the ocular test article at 15, 30, 45, 60, 120, 180, and 240 minutes post-dose. Special attention was afforded to tear film retention and tolerability. Gross examinations include tearing, redness, swelling, and blinking. At 24 hours post dose, animals in Groups 1 and 2 are lightly sedated and the treated eye is thoroughly examined for any presence of the tear film. If any tear film is detected, it is noted in the clinical observations and the remaining film removed. Additional clinical observations are noted as necessary if abnormalities continue past the final observation time point. If any unexpected clinical signs are present, the veterinarian is notified immediately. Animals are restrained manually, chemically (Ketamine or alternative e.g., Telazol, if needed, per Veterinary Guidelines), or mechanically (chair). Dosing is with light sedation. Observations are performed on lightly sedated or awake animals. The test articles are administered to lightly sedated animals (Ketamine 5-15 mg/kg, IM or alternative[e.g., Telazol 5-10 mg/kg, IM]). The time of dose administration is considered as the completion of dosing to the one eye. Once lightly sedated, a single insert test article is placed into the central, lower cul-de-sac of the left eye of all animals with forceps or another appropriate device.

Eye insert disks are composed of 40% HPGuar/40%1 HA/10% PVP/10% PEG and are labelled as TA1 and TA2. TA1 has a diameter of 6 mm and a thickness of 86 microns (std. deviation is 8.4 microns). TA2 has a diameter of 6 mm and a thickness of 108 microns (std. deviation is 8.3 microns). SYSTANE ULTRA® is used as the control.

Observations and Conclusions

Following T=0 dosing, the thinner TA1 inserts were harder to place and they tended to fold once they touched the moisture on the tissue but once situated they lay flat without much trouble. The thicker films, TA2, did not fold and were easy to insert and lay flat immediately on the tissue. Both Groups TA1 and TA2 had mild to moderate tearing after insertion (the animals that received the drops had no tearing). There have been no signs of irritation, no redness, no eye rubbing and no other squinting observed over three hours. After 24 hours, no residual insert material was present in any animal and all of the treated eyes looked acceptable compared to the SYSTANE ULTRA® topical drop, with no redness, swelling or any other signs of irritation.

### Example 7—Human Study of a Lubricant Ocular Insert

In order to assess the biocompatibility, safety, and tolerability of an ocular insert, a study of an embodiment of the present disclosure was tested in a randomized, cross-over design in human participants. Over the course of one study day, a total of three treatments were applied to each participant: 2 ocular inserts and one ocular lubricant drop. Treatments were applied to one eye only, with the fellow untreated eye acting as the control eye.

Study Design:

Ten participants enrolled in the study (5 female, 5 male). The mean age of the participants was 35.5 years (median 33 years, ranging from 23 to 61 years).

The outcome measures for the study were as follows:

Primary outcome variable: Subjective rating of ocular comfort.

Secondary outcome variable: Subjective rating of visual blur, Ocular insert dissolution rate, Investigator rating of handling and non-invasive tear break-up time (NITBUT).

The study was conducted as follows: The study day lasted approximately 9 hours and included a screening and eligibility check, insertion of the first treatment (ocular insert or ocular lubricant drop) into 1 eye, assessments, and eye rinse approximately 2 hours after insertion. After waiting for a minimum of 1 hour, the second treatment was applied to the other eye (eye not previously used) and procedures repeated. There was a wait of a minimum of 1 hour before the final treatment was applied and procedures repeated. For each treatment, ocular comfort and vision ratings were completed: prior to insertion, 5, 15, 30, 60, 90, and 105 minutes after insertion to assess tolerability of the treatment. Tear film assessments were carried out 5, 60, 90 and 105 minutes after insertion. Ocular safety measurements were carried out at screening and after each treatment. At the end of the study day, participants were asked to indicate their treatment preference.

Study Materials:

The two different ocular inserts are as follows:

TABLE 19

| | Ocular Insert 1 | Ocular Insert 2 |
|---|---|---|
| | (Thick insert) | (Thin insert) |
| Ingredients | Hydroxypropyl Guar | Hydroxypropyl Guar |
| | (HPGuar) 40% | (HPGuar) 40% |
| | Hyaluronic Acid (HA) 40% | Hyaluronic Acid (HA) 40% |
| | Polyvinylpyrrolidone/ | Polyvinylpyrrolidone/ |
| | Polyethylene glycol | Polyethylene glycol |
| | (PVP/PEG) 10% | (PVP/PEG) 10% |
| Other | UV sterilised | UV sterilised |
| information | | |
| Diameter | 6 mm circular shape | 6 mm circular shape |
| Thickness | 135-145 μm | 105-115 μm |

Ocular lubricating drops (Systane) were used as a control treatment. The components of the ocular lubricating drops are as follows:

| | |
|---|---|
| Ingredients | Hydroxypropyl Guar (HPGuar), |
| | Polyethylene Glycol (PEG) 400 0.4%, |
| | Propylene Glycol 0.3% |
| Preservative and disinfectant/ | POLYQUAD (polyquaternium-1) |
| cleaning agent | 0.001% w/v |

This study took place over one study day. During the study day, participants were asked to attend five scheduled visits.

Visit 1: Screening and eligibility (0.75 hrs)

Visit 2: Treatment 1 insertion, assessments and removal (2.0 hrs)

Visit 3: Treatment 2 insertion, assessments and removal (2.0 hrs)

Visit 4: Treatment 3 insertion, assessments and removal (2.0 hrs)

Visit 5: Study Exit (0.25 hrs).

A one-hour washout period was applied between visits 2 and 3, and between 3 and 4.

The procedures at each visit are summarized in Table 20

TABLE 20
Study visits and Procedures

| | Screening | Treatment | |
|---|---|---|---|
| | & | insertion & | |
| Measurement & | Eligibility | assessment | Study Exit |
| processes | (0.75 hrs) | (2 hrs/treatment) | (0.25 hrs) |
| Informed Consent | ✓ | | |
| Demographics | ✓ | | |
| Medical history | ✓ | ✓ | |
| VA (logMAR) with | ✓ | ✓ | ✓ |
| spectacles | | | |
| Full slit lamp | ✓ | | ✓ |
| biomicroscopy | | | |
| Partial slit lamp | | ✓ | |
| biomicroscopy | | | |
| Confirm eligibility | ✓ | | |
| Treatment insertion | | ✓ | |
| Treatment removal | | ✓ | |
| Subjective ratings | ✓ | ✓ | |
| (questionnaires) | | | |
| Investigator ratings | | ✓ | |
| (ease of insertion) | | | |
| Tear film meniscus | | ✓ | |
| height | | | |
| Non-invasive tear film | | ✓ | |
| break-up time with a | | | |
| placido disk device | | | |
| Assessment of ocular | | ✓ | |
| insert | | | |

Study Results:

Primary Outcome Variable—Comfort Rating

At each of the time points (prior to insertion, 5, 15, 30, 60, 90, and 105 minutes after insertion), participants were asked the following question, “How would you rate the comfort of your eyes?”.

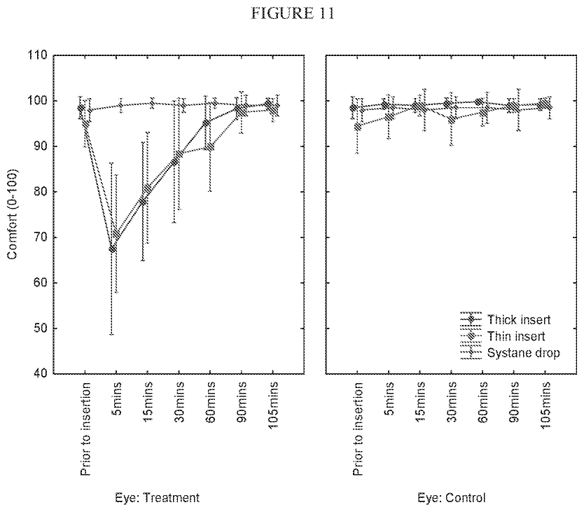

Participants responded using a 0 to 100 scale where 0 indicates “Very poor comfort” and 100 indicates “Excellent comfort”. The results are provided in FIG. 11.

As seen in FIG. 11, there was a statistically significant difference between the inserts and the drop at the 5 and 15-minute time points, with the drop having statistically significant higher comfort ratings. The two inserts performed similarly in terms of comfort ratings, however there was a statistically significant difference at the 60-minute time point, with the comfort of the thick insert rated higher (90 vs 95, p=0.04).

Secondary Outcome Variable

Results of the secondary outcome variables were as follows:

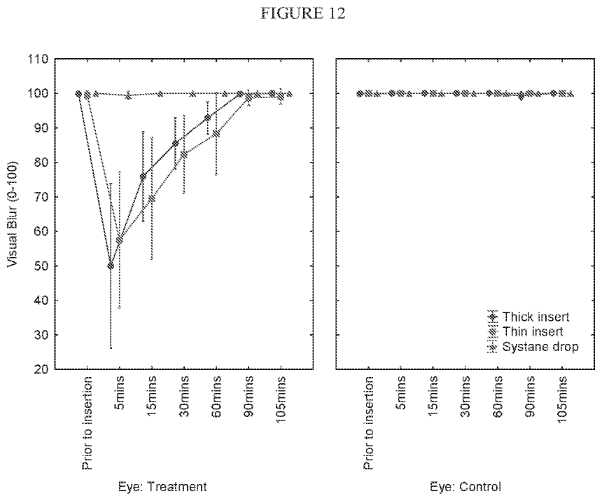

Visual blur—At each of the time points (prior to insertion, 5, 15, 30, 60, 90, and 105 minutes after insertion), participants were asked the following question, “How would you rate the visual blur of your eyes?”. Participants responded using a 0 to 100 scale where 0 indicates “Extremely blurry. Unable to see properly” and 100 indicates “Not blurry at all”. The results are provided in FIG. 12.

As seen in FIG. 12, compared to the drops, both inserts caused statistically significantly lower ratings of visual blur at the 5, 15 and 30-minute time points (p<0.01 or p=0.01). Additionally, the thick insert caused a statistically significant lowering of visual blur ratings at the 60-minute time point (93 vs 100, p=0.01), however there was not a statistically significant difference between the thick and thin insert at 60 minutes (88 vs 93, p=0.28).

Ease of insertion by clinicians—Investigators provided an assessment of the ease of insertion of the ocular inserts during at insertion Ease of insertion was assessed using a 0 to 4 scale in 0.5 steps where 0 indicates “Very easy” and 4 indicates “Very difficult”.

Results are as follows: Thin insert—1.3±0.5, Thick insert—1.6±0.5. The results show that there were no statistically significant difference in the ease of insertion of the two inserts. Further, Inserts were relatively easy to place in eye even with minimal training.

Ocular insert dissolution rate—The degree of dissolution of the ocular inserts were assessed at each time point. At each of the time points (at insertion, 45, 60, 75, 90, and 105 minutes after insertion), Investigators provided an assessment of the degree of dissolution of the ocular inserts. Dissolution grading was done using a 0 to 6 scale, where 0 indicates “No dissolution” and 6 indicates “completely dissolved”.

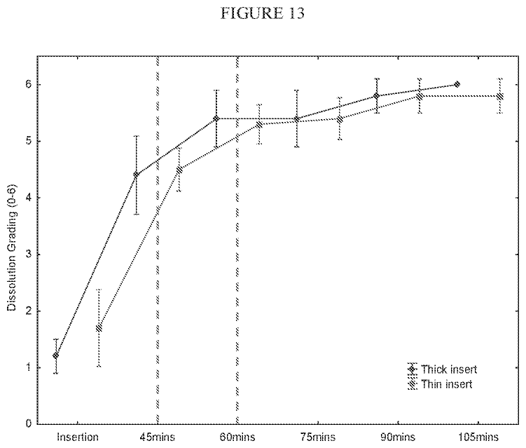

Results are shown in in FIG. 13 and indicate that ˜90% of the lubricant solid material is dissolved between 60-90 min. Further, the data indicate that there were no statistically significant differences in the dissolution grade of the two inserts.

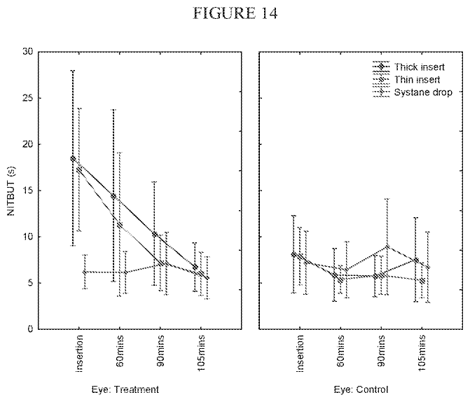

NITBUT—An assessment of the non-invasive tear breakup time (NITBUT) was performed at insertion and 60 minutes, 90 minutes, and 105 minutes after insertion. Results are presented in FIG. 14.

FIG. 14 illustrates that there were statistically significant differences between the treatment eye and the control eye for both inserts at the insertion time point, with the NITBUT of the treatment eye being larger than the control eye (thick insert: 18.52 vs 8.12, p=0.01; thin insert: 17.26 vs 7.93, p<0.01). There were also statistically significant differences with the thick insert between treatment and control eyes at the 60 and 90-minute time points (60 mins: 14.47 vs 5.89, p=0.02; 90 mins: 10.36 vs 5.77, p=0.02).

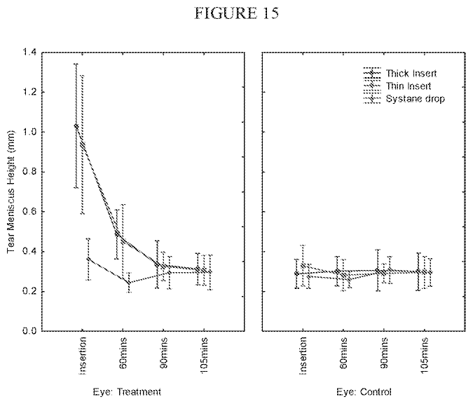

Tear meniscus—Investigators provided an assessment of tear film meniscus height during the treatment at time points and 60 minutes, 90 minutes, and 105 minutes after insertion. Results are presented in FIG. 15.

Other Variables

Ocular irritation—At each of the time points (prior to insertion, 5, 15, 30, 60, 90, and 105 minutes after insertion), participants were asked, “How would you rate the feeling of ocular irritation of your eyes?” Participants responded using a 0 to 100 scale where 0 indicates “Intense feeling of ocular irritation” and 100 indicates “No feeling of ocular irritation at all”.

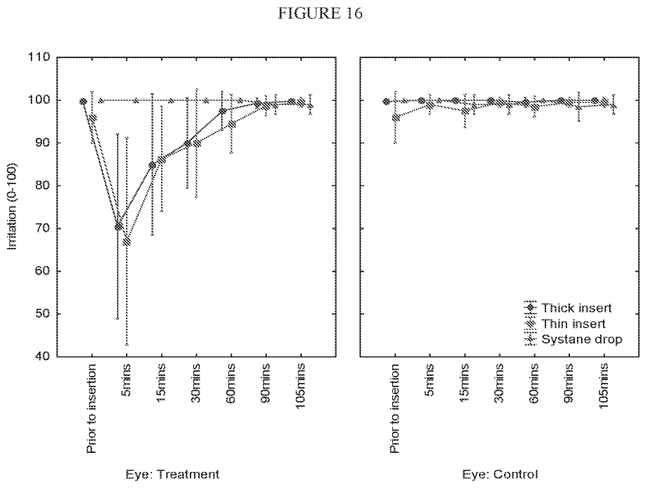

Results are shown in FIG. 16. As seen in FIG. 16, There were statistically significant differences between the treatment eye and the control eye for both inserts at the 5-minute time point, with the ocular irritation rating of the treatment eye being statistically significantly lower (thick insert: 71 vs 100, p=0.01; thin insert: 67 vs 99, p=0.02). There were no statistically significant differences in ocular irritation rating between the two inserts.

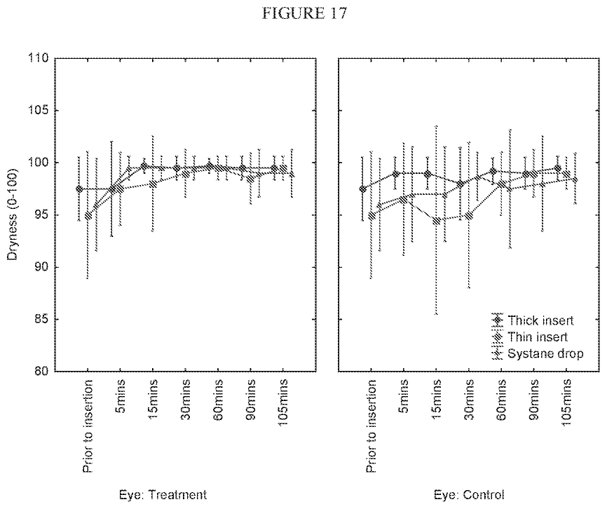

Ocular dryness—At each of the time points (prior to insertion, 5, 15, 30, 60, 90, and 105 minutes after insertion), participants were asked, “How would you rate the dryness of your eyes?” Participants responded using a 0 to 100 scale where 0 indicates “Very dry” and 100 indicates “No dryness at all”. Results are shown in FIG. 17. There were no statistically significant differences between the treatment and control eyes in terms of dryness for any treatment. There were also no differences between inserts.

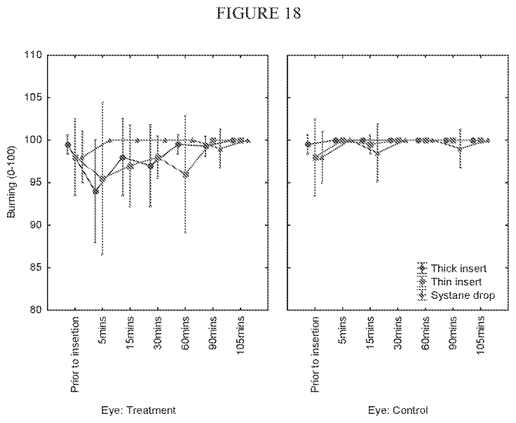

Burning/stinging—At each of the time points (prior to insertion, 5, 15, 30, 60, 90, and 105 minutes after insertion), participants were asked, “How would you rate the burning/stinging sensation of your eyes?” Participants responded using a 0 to 100 scale where 0 indicates “Intense feeling of burning/stinging” and 100 indicates “No burning/stinging at all”. Results are presented in FIG. 18. There were no statistically significant differences between the treatment and control eyes in terms of burning/stinging sensation for any treatment. There were also no differences between inserts.

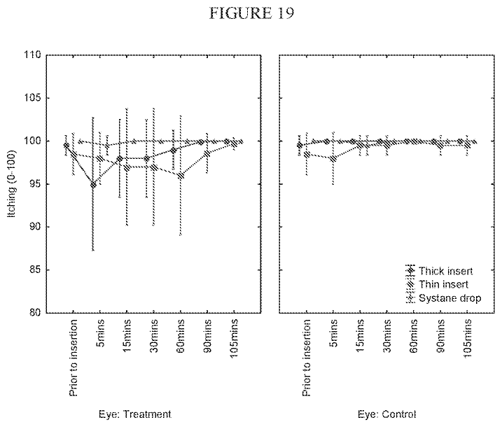

Itching—At each of the time points (prior to insertion, 5, 15, 30, 60, 90, and 105 minutes after insertion), participants were asked, “How would you rate the feeling of itchiness of your eyes?” Participants responded using a 0 to 100 scale where 0 indicates “Intense itching” and 100 indicates “No itching at all”. Results are presented in FIG. 19. As seen in the results, there were no statistically significant differences between the treatment and control eyes in terms of itching for any treatment. There were also no differences between inserts.

High illumination, high contrast visual acuity—Investigators provided an assessment of high contrast, high illumination visual acuity at screening and during the treatment visits at insertion and 60 and 105 minutes after insertion. There were statistically significant differences between treatment and control eyes at the insertion and 60-minute time points, with participants exhibiting a significant reduction in visual acuity. (Insertion: thick insert: 0.05 vs −0.11, p<0.01; thin insert: 0.07 vs −0.10, p=0.01)(60 mins: thick insert: −0.06 vs −0.1, p=0.03; −0.07 vs −0.10, p=0.02). The difference in visual acuity at 60 minutes equates to approximately 2 letters, which may not be considered clinically relevant. Visual acuity had returned to normal by the end of the treatment visit. There were no statistically significant differences in visual acuity between inserts.

Ocular health—Bulbar and limbal hyperemia (redness) and neovascularization were assessed using a 0-4 scale in 0.1 steps (Efron Scale), with 0 indicating normal and 4 indicating severe. There were no clinically relevant differences for any measure of ocular health.

Summary of Results
Primary clinical outcome of subjective comfort and tolerability was achieved. The inserts were tolerated by the participants and the ocular health for all patients during and post-wear was not negatively affected.
There were no statistically significant differences between SYSTANE ULTRA and the inserts for burning, stinging, itching or dryness in the participants.
Inserts did not negatively impact ocular health.
The in vivo rabbit and clinical comfort study data suggest that the device could be a valuable platform for delivery of ocular lubricants and other topical ocular drugs.

### Example 8

Ocular Films Containing 4-(7-Hydroxy-2-isopropyl-4-oxo-4H-quinazolin-3-yl)-benzonitrile

Preparation of Stock Solutions

The following procedure describes the preparation of an 800 g stock solution having the formulation (HA 40/HPGuar 40/PVP10/PEG 400) at a 0.7 g/100 ml concentration with 1.5% 4-(7-hydroxy-2-isopropyl-4-oxo-4H-quinazolin-3-yl)-benzonitrile in a solid form. This procedure results in the preparation of films having a thickness of 110-130 microns, post hydration.

Distilled water (800 ml±50 ml) was transferred from a graduated cylinder into a clean 1000 ml Erlenmeyer flask followed by the addition of hyaluronic acid (2.24 g±0.05 g) and PVP (0.56 g±0.05 g). The flask was placed in a sonicator and attached to an overhead mechanical stirrer. The mixture was sonicated and stirred at room temperature until a viscous, clear, and homogenous solution was obtained (90±30 mins). The speed of the mechanical stirrer was adjusted to 400±50 rpm. HPGuar (2.24 g±0.05 g) was added and the mixture was sonicated and stirred for another 90±30 minutes at room temperature. To the clear, viscous, and homogenous solution, PEG 400 (0.56 g±0.05 g) was added. The mixture was sonicated and stirred for 30 minutes±10 mins at room temperature. To the clear, viscous, and homogenous solution, 4-(7-Hydroxy-2-isopropyl-4-oxo-4H-quinazolin-3-yl)-benzonitrile form B described in U.S. Pat. No. 8,349,852 (84 mg±5 mg) was added as a powder. The mixture was sonicated and stirred for 90±30 minutes at room temperature. The sonicator was then stopped and the mixture was allowed to continue stirring overnight (16 hours±2 hours). The overhead stirring was stopped the next day and the mixture was sonicated for another 30 minutes±10 mins, in order to release any bubbles. The resultant stock solution was used to prepare ocular inserts as described below. The technique described above produced films whereby the original crystalline morphology of 4-(7-hydroxy-2-isopropyl-4-oxo-4H-quinazolin-3-yl)-benzonitrile form B was preserved

Procedure Using a 4-(7-Hydroxy-2-isopropyl-4-oxo-4H-quinazolin-3-yl)-benzonitrile—Ethanol Solution:

The following procedure describes the preparation of an 800 g stock solution having the formulation (HA 40/HPGuar 40/PVP10/PEG 400) at a 0.7 g/100 ml concentration with a 1.5% loading of 4-(7-Hydroxy-2-isopropyl-4-oxo-4H-quinazolin-3-yl)-benzonitrile using a 4-(7-Hydroxy-2-isopropyl-4-oxo-4H-quinazolin-3-yl)-benzonitrile—Ethanol Solution. This procedure results in the preparation of films having a thickness of 110-130 microns after hydration.

Distilled water (800 ml±50 ml) was transferred from a graduated cylinder into a clean 1000 ml Erlenmeyer flask followed by the addition of hyaluronic acid (2.24 g±0.05 g) and PVP (0.56 g±0.05 g). The flask was placed in a sonicator and attached to an overhead mechanical stirrer. The mixture was sonicated and stirred at room temperature until a viscous, clear, and homogenous solution was obtained (90±30 mins). The speed of the mechanical stirrer was adjusted to 400±50 rpm. HPGuar (2.24 g±0.05 g) was added and the mixture was sonicated and stirred for another 90±30 minutes at room temperature. To the clear, viscous, and homogenous solution, PEG 400 (0.56 g±0.05 g) was added. The mixture was sonicated and stirred for 30 minutes±10 min at room temperature. To the clear, viscous, and homogenous solution, 4-(7-hydroxy-2-isopropyl-4-oxo-4H-quinazolin-3-yl)-benzonitrile (84 mg±5 mg) dissolved in 10 ml±1 ml ethanol was added. The mixture was sonicated and stirred for 30 minutes±10 min at room temperature. The overhead stirring was then stopped and the mixture was sonicated for another 30 minutes±10 min in order to release any bubbles. The resultant stock solution was used to prepare ocular inserts as described below.

Film Casting Procedure:

All films were prepared by filling a petri dish (150 mm diameter×15 mm height) with 150 g±10 g of the stock solution (prepared by either dissolving or dispersing the solid pharmaceutically active agent in the polymer mixture) and placing the petri dish in an evaporation chamber for 40-48 hours.

The dimensions of the evaporation chamber are 3 feet×2 feet×2 feet (height×depth×width). The chamber was equipped with an exhaust fan to provide 110 cfm of air flow through the chamber. The temperature inside the chamber was controlled at 27-30° C. during the evaporation process.

Ocular Film Hydration Procedure:

The film was cut into 6 mm disks with a disk cutter. The thickness of each disk was measured before hydration (the thickness is 50-70 microns). Two disks were placed into a pouch and 2-3 μL sterilized deionized water was introduced into one corner of the pouch. The pouch was then sealed using a heat sealer. The films were then subjected to gamma sterilization.

Procedure Using the Sonication/Stirring Method:

Measurement of Various Film Properties for Ocular Films with 4-(7-Hydroxy-2-isopropyl-4-oxo-4H-quinazolin-3-yl)-benzonitrile:

A series of physical properties were measured for ocular films with 4-(7-hydroxy-2-isopropyl-4-oxo-4H-quinazolin-3-yl)-benzonitrile. The procedures for each property measurement are outlined below and the results are summarized in Table 21.

Thickness and Weight:

Dry film disks having 6 mm diameters were cut with a disk cutter, weighed, and measured for thickness with a digital micrometre. After hydration treatment, each disk was measured for thickness.

Moisture Absorption:

Inside a Class 6 cleanroom, disks having 2 cm diameters were cut with a disk cutter and weighed. Each disk was placed in a hydration chamber providing 80% internal moisture. After 72 hours of hydration exposure, the disks were removed from the chamber and weighed. The % moisture absorption was determined [% moisture absorption=final weight−initial weight/initial weight×100].

Moisture Loss:

Inside a Class 6 cleanroom, disks having 2 cm diameter were cut with a disk cutter and weighed. Each disk was placed in a chamber containing anhydrous calcium chloride. After 72 hours drying, the disks were removed from the chamber and weighed. % Moisture loss was calculated.

Folding Endurance:

3×3 cm² cut films were folded in half, down the middle, and then opened. The fold-opening cycle was repeated until film breakage occurred. Record the folding time once breakage occurs (if no breakage occurs after folding 100 times, record as >100).

Dissolution Time:

6 mm diameter film disks were cut with a disk cutter and placed in separate 4 ml glass vials. DI water (2 ml) was added to each vial and the vials capped. Each vial was shaken by hand until the disks had dissolved by visual inspection. The dissolution time was recorded.

Formulation pH:

After obtaining a homogeneous formulation solution, the pH of the solution was measured using an OakIon pH meter.

Mechanical Tests:

1-1.5 cm×4 cm film strips were cut with a sharp knife and then hydrated for 36-48 hours in separate, sealed aluminum foil bags containing 60 μL added DI water. The resulting hydrated film strips were then subjected to mechanical tests (Young's modulus and % Elongation at break) using an Instron.

TABLE 21
Physical Characterization (Funnel Test) Results for Ocular Films with 1.5%
4-(7-Hydroxy-2-isopropyl-4-oxo-4H-quinazolin-3-yl)-benzonitrile (referred to as
Compound A for the purposes of Table 21) as compared to Ocular Films without
4-(7-Hydroxy-2-isopropyl-4-oxo-4H-quinazolin-3-yl)-benzonitrile

| | | Ocular Film with | Ocular Film | |
|---|---|---|---|---|
| | | 1.5% Compound A | with 1.5% | Ocular Film |
| Test | | (sonication/stirring | Compound A | without |
| Parameter | Test Method | method) | (solution method) | Compound A |
| Morphology | Visual Appearance | Hazy and uniform | Almost clear | Clear and |
| | | film, hydrated film, | film, hydrated | transparent |
| | | white | film, hazy | film |
| Thickness | Digital Caliper | 0.05-0.07 mm (dry) | 0.05-0.07 mm (dry) | 0.05-0.07 mm (dry) |
| | | 0.126 mm (wet) | 0.115 mm (wet) | 0.1-0.15 mm (wet) |
| Weight | Microbalance | 2.69 mg (dry) | 3.02 mg (dry) | 2.3-3.2 mg (dry) |
| | (6 mm disks) | | | |
| Moisture | Saturated | 19% | 20% | 19.51% |
| Absorption | Ammonium chloride | | | |
| | solution Chamber | | | |
| Moisture | Calcium Chloride | 6.23% | 6.69% | 4.38% |
| Loss | Chamber Method | | | |
| Flex | Cycle bending at | >100 | >100 | >100 |
| Endurance | 90° angle until | | | |
| | cracking or breaking | | | |
| Dissolution | Solubility in DI | 3.46 min, | 2.73 min, | 1-4 min, |
| time in DI | water and pH | pH 7.24 | pH 7.24 | pH 6.8-7.5 |
| water & pH | measurement | | | |
| of solution | | | | |
| % | Instron Test Method | 122.68% (wet) | 128.17% (wet) | 148% (wet) |
| Elongation | | | | |
| at Break | | | | |
| Young's | Instron Test Method | 0.467 MPa (wet) | 0.341 MPa (wet) | 0.2 MPa (wet) |
| Modulus | | | | |

Recovery of Compound a from Hydrated Films

Compound A was extracted from a representative number of hydrated films to evaluate the stability of Compound A in hydrated films. After extraction for three days with a Acetonitrile:methanol (1:2) solvent mixture, the recovery rate of Compound A from the films was in the range of 95-99% of original amount of Compound A present in the films.

Stability of Drug Loaded Films after Gamma Sterilization

The Compound A loaded gamma sterilized films were tested for visual changes, wet thickness, dissolution rate, solution pH, mechanical testing and Compound A recovery before and after gamma sterilization. The results are shown in the Table below and indicate that the ocular films are able to retain their properties even after gamma sterilization.

| | Before gamma | | After gamma | |
|---|---|---|---|---|
| | sterilization | | sterilization | |
| Visual characteristic | Hazy | | Hazy | |
| Wet thickness | 0.12 | mm | 0.122 | mm |
| Dissolution rate | 2.72 | mins | 2.90 | mins |
| pH | 6.86 | | 7.14 | |
| Elongation | 88.5% | | 151.35 | |
| Young's Modulus | 0.128 | psi | 0.075 | psi |

| | | |
|---|---|---|
| Compound A recovery | 111.33% | 104.34% |

Stability Testing of Drug Loaded Films

Compound A loaded films, prepared as described above were tested for stability after storage at 25° C. and 30° C. for two months. Results are shown in the Table below.

| | | | | Film loaded with | | | |
|---|---|---|---|---|---|---|---|
| | | | | Compound A | | Film loaded with | |
| Storage | | Blank film | | (ethanol | | Compound A | |
| condition | Testing criterion | (no drug) | | solution method) | | (powder method) | |
| 2 month | Visual characteristic | Clear and | | Almost clear | | Hazy | |
| at 25° C. | | transparent | | | | | |
| | Wet thickness | 0.125 | mm | 0.118 | mm | 0.115 | mm |
| | Dissolution rate | 1.4 | mins | 1.35 | mins | 2.02 | mins |
| | pH | 7.42 | | 7.44 | | 7.43 | |
| | Elongation | 105.14% | | 128.56% | | 91.13% | |
| | Young's Modulus | 0.155 | psi | 0.087 | psi | 0.054 | psi |
| | Compound A recovery | NA | | 109.33% | | 108.72% | |
| 2 months | Visual characteristic | Clear and | | Almost clear | | Hazy | |
| at 30° C. | | transparent | | | | | |
| | Wet thickness | 0.118 | mm | 0.128 | mm | 0.103 | mm |
| | Dissolution rate | 1.18 | mins | 0.71 | mins | 1.09 | mins |
| | pH | 7.32 | | 7.41 | | 7.47 | |
| | Elongation | 55.95% | | 52.03% | | 76.06% | |
| | Young's Modulus | 0.159 | psi | 0.104 | psi | 0.162 | psi |

| | | | |
|---|---|---|---|
| Compound A recovery | NA | 94.67% | 93.29%* |

As seen in the Table above, the drug-loaded polymeric eye inserts (i.e., ocular film) were able to maintain acceptable physical characteristics and acceptable drug recovery after storage at 25° C. and 30° C. for two months.

In addition, the films passed sterility tests (USP <71> method) and a bacterial endotoxin test (USP <85> method) after storage at 1 month and 2 months at 25° C. and 30° C.

XRD Results (Dry Films were Used to Run XRD):

Films that were prepared using the sonication/stirring method showed that the original crystalline morphology of 4-(7-hydroxy-2-isopropyl-4-oxo-4H-quinazolin-3-yl)-benzonitrile was preserved. Films that were prepared via the method using an ethanolic solution of 4-(7-Hydroxy-2-isopropyl-4-oxo-4H-quinazolin-3-yl)-benzonitrile did not contain original crystalline morphology (see FIG. 20).

Summary of the Results
Polymorph stability of 4-(7-hydroxy-2-isopropyl-4-oxo-4H-quinazolin-3-yl)-benzonitrile form B was unaffected by the sonication/stirring method as the crystal morphology remained unchanged when incorporated into the polymeric eye insert.
Physicochemical and mechanical stability properties of the polymeric eye inserts were preserved upon incorporation of a pharmaceutically active agent, e.g., 4-(7-hydroxy-2-isopropyl-4-oxo-4H-quinazolin-3-yl)-benzonitrile (e.g., Table 21).
The physicochemical and mechanical properties suggest that the polymeric eye insert could be a valuable platform for the delivery of one or more pharmaceutically active agents, e.g., 4-(7-hydroxy-2-isopropyl-4-oxo-4H-quinazolin-3-yl)-benzonitrile.
   The data presented here indicate that 4-(7-Hydroxy-2-isopropyl-4-oxo-4H-quinazolin-3-yl)-benzonitrile (e.g., form B) can be incorporated into a thin film formulation while still maintaining overall acceptable physical properties.

### Rabbit Ocular Pharmacokinetic Study

An exemplary ocular polymeric insert formulation according to the present invention was tested in male New Zealand white rabbits. A polymeric insert formulation containing 0.05 mg of 4-(7-hydroxy-2-isopropyl-4-oxo-4H-quinazolin-3-yl)-benzonitrile was compared to a suspension formulation having a concentration of 1.5% of 4-(7-hydroxy-2-isopropyl-4-oxo-4H-quinazolin-3-yl)-benzonitrile (about 0.525 mg dose/eye). The testing was conducted using the following procedure: the test suspension (35 μL) or polymeric insert formulation was administered to each eye at the central or superior part of the cornea via a positive displacement pipette and allowed to spread across the surface of the eye (for suspension) or administered to the lower cul-de-sac (for polymeric eye insert formulation). After the dose was administered, the eye was allowed to close naturally. Each animal was restrained for approximately 1 minute to prevent rubbing of the. An Elizabethan collar was placed on all animals prior to returning to their cage, and was removed at approximately 2 hours postdose.

Sample Collection

Blood

Two animals/group/time point were sacrificed 0.5, 1, 2, 4, 8, 12, and 24 hours postdose. Blood (approximately 5 mL) was collected into tubes containing K₂EDTA via cardiac puncture from all animals at sacrifice. Blood was maintained on wet ice or in chilled cryoracks prior to centrifugation to obtain plasma.

Ocular Tissues

At the time of sacrifice, following blood collection, both eyes were enucleated, rinsed thoroughly with saline, and the aqueous humor, conjunctiva, and cornea were collected fresh.

Each of the biological samples were analyzed for concentrations of 4-(7-hydroxy-2-isopropyl-4-oxo-4H-quinazolin-3-yl)-benzonitrile using an established liquid chromatography/mass spectrometry (LC-MS/MS) method. Mean pharmacokinetic parameters are shown in the following Table.

| | | Aqueous | | |
|---|---|---|---|---|
| | | humor | Conjunctiva | Cornea |
| | time | ng/ml | ng/g | ng/g |
| Suspension | 0.5 | 926 ± 198 | 1648 ± 252 | 4863 ± 503 |
| | 1 | 253 ± 45.0 | 607 ± 180 | 1413 ± 358 |
| | 2 | 71.7 ± 32.3 | 332 ± 189 | 875 ± 652 |
| | 4 | 13.2 ± 8.13 | 119 ± 172 | 142 ± 144 |
| | 8 | 1.15 | 9.12 ± 4.28 | 19.6 ± 3.59 |
| | 12 | 0.0 | 7.24 | 14.0 ± 5.90 |
| | 24 | 0.0 | 10.2 ± 4.86 | 17.8 ± 11.0 |
| Insert | 0.5 | 1019 ± 113 | 1548 ± 336 | 6308 ± 778 |
| | 1 | 494 ± 154 | 510 ± 98.1 | 2138 ± 566 |
| | 2 | 90.0 ± 8.94 | 52.1 ± 19.1 | 361 ± 71.5 |
| | 4 | 4.31 ± 2.14 | 16.1 ± 8.88 | 77.3 ± 56.8 |
| | 8 | 1.40± | 16.5 ± 7.20 | 29.5 ± 18.6 |
| | 12 | 0.0 | 19.4 ± 4.10 | 41.4 ± 32.8 |
| | 24 | 0.0 | 21.0 | 14.8 |

| | | | Cmax | AUClast | AUCinf | | Clast |
|---|---|---|---|---|---|---|---|
| | | Tmax | ng/mL | h*ng/ | h*ng/ | Tlast | ng/mL |
| Tissue | Formulation | h | or g | mL or g | mL or g | h | or g |
| Aqueous | Insert | 0.5 | 1020 | 1030 | 1030 | 8 | 1.40 |
| humor | | | | | | | |
| | Suspension | 0.5 | 926 | 802 | 804 | 8 | 1.15 |
| Con- | Insert | 0.5 | 1550 | 1630 | 1900 | 24 | 21.0 |
| junctiva | Suspension | 0.5 | 1650 | 2290 | 2350 | 24 | 10.2 |
| Cornea | Insert | 0.5 | 6310 | 6070 | 6270 | 24 | 14.8 |
| | Suspension | 10.5 | 4860 | 5530 | 5620 | 24 | 17.8 |
| Plasma | Insert | 0.5 | 9.33 | 9.04 | n.a. | 2 | 0.216 |
| | Suspension | 0.5 | 15.9 | 12.4 | 12.8 | 4 | 0.275 |

As seen from the results above, both the polymeric insert formulation having an approximately ten fold lower dose of drug shows similar pharmacokinetic properties to a suspension formulation. Accordingly, an polymeric insert formulation according to the present invention may be able to achieve higher drug levels for ophthalmically active agents in the eye and extended release of such drugs.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. All of the publications and patent applications and patents cited in this specification are herein incorporated in their entirety by reference.

1. A polymeric eye insert, the insert comprising:
one or more mucoadhesive polymers that are biocompatible with the ocular surface and tear film of the eye; and
one or more pharmaceutically active agent.

2. The polymeric eye insert of claim 1, wherein the one or more mucoadhesive polymers are selected from the group consisting of:
hyaluronic acid or salts thereof, hydroxypropylmethylcellulose (HPMC), methylcellulose, tamarind seed polysaccharide (TSP), guar, hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and combinations thereof.

3. The polymeric eye insert of claim 1 wherein the one or more mucoadhesive polymers are HP guar, hyaluronic acid, or sodium hyaluronate.

4. The polymeric eye insert of any of the preceding claims, wherein the one or more mucoadhesive polymers are present in an amount of from about 50% to about 99% w/w, about 60% to about 95% w/w, about 70% to about 90% w/w, or about 80% to about 90% w/w of the polymeric eye insert.

5. The polymeric eye insert of any of the preceding claims, wherein the one or more mucoadhesive polymers are present in an amount of about 75%, about 80%, about 85%, about 90%, or about 95% w/w.

6. The polymeric eye insert of claim 3 or 4 or 5 further comprising a plasticizer or softener.

7. The polymeric eye insert of claim 6 wherein the plasticizer or softener is selected from the group consisting of:
polyethylene glycol (PEG), a PEG derivative, water, Vitamin E, and triethyl citrate.

8. The polymeric eye insert of claim 6 or 7, wherein the plasticizer or softener is present in an amount of from about 2% to about 30% w/w, about 5% to about 25% w/w, about 5% to about 20% w/w, or about 5% to about 15% w/w of the polymeric eye insert.

9. The polymeric eye insert of claim 6 or 7 or 8, wherein the plasticizer or softener is present in an amount of about 5%, about 7%, about 10%, or 12%, or about 15%, w/w.

10. The polymeric eye insert of any of claims 6 to 9, wherein the plasticizer or softener is PEG.

11. The polymeric eye insert of any one of the preceding claims, wherein the insert is comprised of approximately 40% HP guar, approximately 10% PVP, approximately 40% sodium hyaluronate, and approximately 10% PEG.

12. The polymeric eye insert of any of the preceding claims, further comprising 1-200 ppm menthol.

13. The polymer eye insert of any of the preceding claims, further comprising 20-100 ppm menthol.

14. The polymeric eye insert of any of the preceding claims wherein the one or more pharmaceutically active agents is selected from the group consisting of drugs used to treat the eye.

15. The polymeric eye insert of any of the preceding claims wherein the one or more pharmaceutically active agents is present in the polymeric eye insert at a concentration of about 0.01-10% w/w or in an amount of from about 0.01 mg to about 10 mg.

16. The polymeric eye insert of claim 14 or 15, wherein the pharmaceutically active agent is 4-(7-hydroxy-2-isopropyl-4-oxo-4H-quinazolin-3-yl)-benzonitrile, 1,1-dioxo-hexahydro-1lambda*6*-thiopyran-4-yl)-{(S)-3-[1-(6-methoxy-5-methyl-pyridin-3-yl)-2,3-dihydro-1H-pyrido[3,4-b][1,4]oxazin-7-yloxy]-pyrrolidin-1-yl}-methanone, 1-{(S)-3-[4-(6-methanesulfonyl-5-methyl-pyridin-3-yl)-3,4-dihydro-2H-benzo[1,4]oxazin-6-yloxy]-pyrrolidin-1-yl}-2-methoxy-ethanone, or {(S)-3-[4-(6-chloro-5-methoxy-pyridin-3-yl)-3,4-dihydro-2H-benzo[1,4]oxazin-6-yloxy]-pyrrolidin-1-yl}-(1,1-dioxo-hexahydro-1lambda*6*-thiopyran-4-yl)-methanone, lipoic acid choline ester, or salts thereof.

17. The polymeric eye insert of claim 14 or 15, wherein the pharmaceutically active agent is 4-(7-hydroxy-2-isopropyl-4-oxo-4H-quinazolin-3-yl)-benzonitrile or salts thereof.

18. The polymeric eye insert of claim 17, wherein the 4-(7-hydroxy-2-isopropyl-4-oxo-4H-quinazolin-3-yl)-benzonitrile or salts thereof is present in an amount of about 0.5% w/w to about 3.5% w/w.

19. The polymeric eye insert of claim 18, wherein the 4-(7-hydroxy-2-isopropyl-4-oxo-4H-quinazolin-3-yl)-benzonitrile or salts thereof is present in an amount of about 0.5% w/w, about 1.0% w/w, about 1.5% w/w, about 2.0% w/w, about 2.5% w/w, about 3.0% w/w, or about 3.5% w/w or wherein the 4-(7-hydroxy-2-isopropyl-4-oxo-4H-quinazolin-3-yl)-benzonitrile or salts thereof is present in an amount from about 0.01 mg to about 5 mg, from about 0.01 mg to about 1 mg, from about 0.01 mg to about 0.5 mg, from about 0.01 mg to about 0.1 mg, from about 0.01 mg to about 0.08 mg per polymeric eye insert. In particular embodiments, the polymeric eye insert includes 4-(7-hydroxy-2-isopropyl-4-oxo-4H-quinazolin-3-yl)-benzonitrile or salts thereof in an amount of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.15 mg, about 0.2 mg, about 0.25 mg, about 0.3 mg, about 0.35 mg, about 0.4 mg, about 0.45 mg, or about 0.5 mg per polymeric eye insert.

20. The polymeric eye insert of claims 17-19, wherein the 4-(7-hydroxy-2-isopropyl-4-oxo-4H-quinazolin-3-yl)-benzonitrile is present as crystal form B, characterized by an X-ray diffraction pattern having three or more peaks at 2θ values selected from 9.3° 10.6° 14.4°±0.2 °2 θ, when recorded using CuK_{α} radiation.

21. The polymeric eye insert of any of the preceding claims, wherein upon insertion in the cul-de-sac of the eye, tear film thickness does not return to pre-insertion thickness until approximately two hours after insertion.

22. The polymeric eye insert of any of the preceding claims, wherein upon insertion in the cul-de-sac of the eye, tear film thickness increases up to at least two hours post-insertion.

23. The polymeric eye insert of any of the preceding claims, wherein the insert shape is suitable for insertion into the eye.

24. The polymeric eye insert of any of the preceding claims, wherein the insert shape is a film, a rod, a sphere, or an irregular shape having a maximum size in any single dimension of 5-7 mm.

25. The polymeric eye insert of any of the preceding claims, wherein the polymeric eye insert has a water content of about 1% to about 50% w/w, in particular about 30-40% w/w.

26. The polymeric eye insert of any of claims 23 to 25, wherein said insert has a circular shape about 5 mm in diameter, a thickness of 50-400 μm, and a water content of 1% to 50% w/w.

27. The polymeric eye insert of claim 26, wherein said insert has a thickness of about 150-250 μm, and a water content of 30 to 50% w/w.

28. The polymeric eye insert of claim 3, wherein said HP guar has a molecular weight of 2 to 3 million Daltons and said sodium hyaluronate has a molecular weight of 0.1 to 1 million Daltons.

29. The polymeric eye insert according to claim 1, wherein upon insertion of the polymeric eye insert in the cul-de-sac of the eye, the thickness of the tear film increases for at least 30 minutes post-insertion.

30. The polymeric eye insert of any of the preceding claims, wherein the polymeric eye insert dissolves over an extended duration of time after insertion into the eye.

31. The polymeric eye insert of any of the preceding claims, wherein the polymeric eye insert dissolves within about 1 to 2 hours, 1 to 3 hours, 1 to 4 hours, 1 to 5 hours, 1 to 6 hours, 1 to 7 hours, 1 to 8 hours, 1 to 9 hours, or 1 to 10 hours after insertion into the eye.

32. The polymeric eye insert of any of the preceding claims, wherein the polymeric eye insert dissolves within about 60 to 90 minutes after insertion into the eye.

33. The polymeric eye insert of any of the preceding claims, wherein the polymeric eye insert dissolves in about 60, about 90, about 120 minutes, about 3 hours, about 4 hours, about 5 hours, about 6 hours, about 7 hours, about 8 hours, about 9 hours, or about 10 hours after insertion into the eye.

34. The polymeric eye insert of any of the preceding claims, wherein the polymeric eye insert has a thickness of about 50-250 μm, particularly of about 70-150 μm, when dissolved within about 60 to 120 minutes after insertion into the eye.

35. The polymeric eye insert of claim 34, wherein the polymeric eye insert has a thickness of about 90 μm when dissolved within about 60 to 120 minutes after insertion into the eye.

36. The polymeric eye insert of any of the preceding claims, wherein the polymeric eye insert has a Young's modulus of less than about 0.6 MPa as determined using the Instron test method.

37. The polymeric eye insert of claim 36, wherein the polymeric eye insert has a Young's modulus of about 0.2-0.5 MPa as determined using the Instron test method.

38. The polymeric eye insert of claim 36 or 37, wherein the polymeric eye insert has a Young's modulus of less than about 0.6 MPa, particularly about 0.2-0.5 MPa, such that the % elongation at break is between about 120-150% as determined using the Instron test method.

39. A method of providing extended drug delivery or prolonging exposure of a pharmaceutically active agent to the eye, by administering the polymeric eye insert according to any of claims 1 to 38, to a patient in need thereof.

40. The method of claim 39, wherein the pharmaceutically active agent is as defined in any of claims 16 to 20.

41. A method of making a polymeric eye insert of any of claims 1 to 38, comprising the step of preparing a mixture comprising the one or more mucoadhesive polymers, optionally, the plasticizer or softener, and one or more pharmaceutically active agent in a suitable first solvent.

42. The method according to claim 41, wherein the mixture is stirred and/or sonicated.

43. The method of claim 41 or 42, wherein the pharmaceutically active agent is as defined in any of claims 16 to 20.

44. A method of treating or reducing the symptoms of an ocular disorder, which comprises applying the polymeric eye insert of any one of claims 1-38 to the cul-de-sac of the eye.

45. A method according to claim 44, wherein said ocular disorder is selected from the group consisting of ocular pain, myopia, presbyopia, dry eye, glaucoma, allergy, ocular hyperemia, inflammation, dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, cornepal erosions or abrasions (including recurrent corneal erosions or abrasions), ocular surface diseases, blepharitis, graft vs host disease, meibomitis, conjunctivochalasis, keratopathis (including herpetic keratopathy, filamentary keratopathy, band or bullous keratopathy, exposure keratopathy), keratitis (including herpes simplex virus keratitis), iritis, and episclentis.

46. The method according to claim 44 or 45, wherein the ocular disorder is ocular surface pain.

47. The method of claim 46, wherein the ocular surface pain is due to dry eye disease, Sjogren's Syndrome, conjunctivitis (including keratoconjuctivitis, vernal keratoconjunctivitis, allergic conjunctivitis), Map-Dot-Fingerprint Dystrophy, acanthamoeba, fibromyalgia, Meibomian gland dysfunction, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), epithelial basement membrane dystrophy, corneal erosions or abrasions (including recurrent corneal erosions or abrasions), ocular surface diseases, blepharitis, graft vs host disease, meibomitis, glaucoma, conjunctivochalasis, keratopathis (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, neuralgia, xerophthalmia, patients recovering from neurotrophic keratitis, or ocular pain persisting for at least three months after photorefractive keratectomy (PRK) surgery or laser-assisted in situ keratomileusis (LASIK) surgery.

48. A polymeric eye insert according to any one of claims 1 to 38, for use in the treatment of an ocular disorder, which comprises applying the polymeric eye insert of any one of claims 1-38 to the cul-de-sac of the eye.

49. The polymeric eye insert for use according to claim 48, wherein said ocular disorder is selected from the group consisting of ocular pain, myopia, presbyopia, dry eye, glaucoma, allergy, ocular hyperemia, and inflammation.

50. The polymeric eye insert for use according to claim 48 or 49, wherein the ocular disorder is ocular surface pain.

51. The polymeric eye insert for use according to claim 50, wherein the ocular surface pain is as defined in claim 47.

52. A pharmaceutical composition in the form of a polymeric eye insert according to any one of claims 1 to 38, for use in the treatment of an ocular disorder as defined in any one of claims 45 to 47, 49 to 51.

53. Use of a polymeric eye insert according to any of claims 1 to 38 in a method as defined by any one of claims 44 to 47.
